(19) United States
(12) Patent Application Publication
(10) Pub. No.: US 2020/0063119 A1
(43) Pub. Date: Feb. 27, 2020

(54) IN VITRO DNA WRITING FOR INFORMATION STORAGE

(71) Applicant: Massachusetts Institute of Technology, Cambridge, MA (US)

(72) Inventors: Timothy Kuan-Ta Lu, Cambridge, MA (US); Fahim Farzadfard, Boston, MA (US)

(73) Assignee: Massachusetts Institute of Technology, Cambridge, MA (US)

(21) Appl. No.: 16/548,143

(22) Filed: Aug. 22, 2019

Related U.S. Application Data
(60) Provisional application No. 62/721,197, filed on Aug. 22, 2018.

(51) Int. Cl. C12N 15/10; G16B 20/00; G16B 40/10; G16B 50/20; G16B 50/30; G16B 50/40; G16B 50/50; B01J 19/00

(52) U.S. Cl. CPC C12N 15/1031; G16B 20/00; G16B 40/10; G16B 50/20; G16B 50/30; B01J 2219/00722; G16B 50/50; B01J 19/0046; C12N 15/1006; B01J 2219/00608; B01J 2219/00623; G16B 50/40

(57) ABSTRACT

Provided herein are compositions, systems, and methods for information (e.g., artificial or digital information) recording and storage in nucleic acids (e.g., DNA). Information can be recorded and stored on pre-synthesized storage medium.

### RELATED APPLICATION

This application claims the benefit under 35 U.S.C. § 119(e) of U.S. Provisional Application No. 62/721,197, filed Aug. 22, 2018, and entitled “IN VITRO DNA WRITING FOR INFORMATION STORAGE,” the entire contents of which are incorporated herein by reference.

### FEDERALLY SPONSORED RESEARCH

This invention was made with Government support under Grant No. CCF1521925 awarded by the National Science Foundation (NSF), and under Grant No. P50 GM098792 awarded by National Institutes of Health. The Government has certain rights in the invention.

### BACKGROUND

Nucleic acids (e.g., DNA) can be used as storage medium for recording and storing information. Synthesizing the nucleic acids (e.g., DNA) can be costly if a new storage medium is required every time new information needs to be recorded.

### SUMMARY

Provided herein, in some aspects, are systems and methods for in vitro information recording and storage using nucleic acids (e.g., DNA) as storage medium. Using the compositions and methods described herein, information can be record with nucleotide precision. Components of the information storage systems described herein include, in some embodiments, a storage medium, address molecules that target the nucleotides in the storage medium, and modifying enzymes that use the address molecules to target and modify the nucleotides in the storage medium. In some embodiments, the compositions and methods described herein can be used in a high throughput format, e.g., in conjunction with a “printer” that is capable of spotting, with high resolution, the address molecule and modifying enzyme onto suitable support medium (e.g., paper) that has been preloaded with the storage medium. The composition and methods described herein are particular useful when low-cost nucleic acid (e.g., DNA) synthesis is not available.

Accordingly, some aspects of the present disclosure provide methods of storing information, including:

(i) providing a storage medium containing a plurality of nucleic acid molecules, each nucleic acid molecule containing one or more information storage regions, each information storage region containing a write address followed by a read address; and

(ii) contacting, in vitro, the storage medium with:
(a) a modifying enzyme containing a DNA binding domain fused to a base editing enzyme that edits one or more target nucleotides in the write address of the plurality of nucleic acid molecules, and
(b) a plurality of guide RNAs (gRNAs), each gRNA containing a specificity determining sequence (SDS) that is complementary to one type of information storage region in the plurality of nucleic acid molecules;

wherein the contacting results in the editing of the one or more target nucleotides in the write address of the plurality of nucleic acid molecules.

In some embodiments, the DNA binding domain is a catalytically-inactive Cas9 (dCas9) or a Cas9 nickase (nCas9). In some embodiments, the DNA binding domain is a catalytically-inactive Cpf1 (dCpf1).

In some embodiments, the plurality of nucleic acid molecules are isolated genomic DNA molecules. In some embodiments, the isolated genomic DNA molecules are isolated bacterial genomic DNA. In some embodiments, the plurality of nucleic acid molecules are plasmids.

In some embodiments, the plurality of nucleic acid molecules are synthetic oligonucleotides. In some embodiments, each synthetic oligonucleotide further contains a sequencing adaptor. In some embodiments, each of the plurality of nucleic acid molecules further contains a protospacer adjacent motif (PAM) following each information storage region. In some embodiments, the plurality of nucleic acid molecules do not each contain a PAM following each information storage region, and the method further includes contacting the storage medium with a PAM-presenting oligonucleotide (PAMmer).

In some embodiments, the a base editing enzyme is a cytidine deaminase and the write address contains one or more deoxycytidines. In some embodiments, the contacting results in a deoxycytidine to thymidine mutation.

In some embodiments, the a base editing enzyme is an adenosine deaminase and the write address contains one or more deoxyadenosines. In some embodiments, the contacting results in a deoxyadenosine to deoxyguanosine mutation.

In some embodiments, the method is carried out in a high-throughput manner.

In some embodiments, the method described herein further includes: (iii) detecting the editing of the one or more target nucleotides. In some embodiments, the detecting is via sequencing.

Other aspects of the present disclosure provide methods of storing information, including:

(i) providing a support medium containing a plurality of spots, each spot containing a storage medium containing a plurality of nucleic acid molecules, each nucleic acid molecule containing one or more information storage regions, each information storage region containing a write address followed by a read address, wherein different spots have different nucleic acid molecules; and

(ii) depositing using a printing device onto the plurality of spots on the support medium:
(a) a modifying enzyme containing a DNA binding domain fused to a base editing enzyme that edits one or more target nucleotides in the write address of the plurality of nucleic acid molecules, and
(b) a plurality of guide RNAs (gRNAs), each gRNA containing a specificity determining sequence (SDS) that is complementary to one type of information storage region in the plurality of nucleic acid molecules, wherein the gRNA deposited onto each spot is different;

wherein the contacting results in the editing of the one or more target nucleotides in the write address of the plurality of nucleic acid molecules, and wherein nucleic acid molecules in different spots have different editing patterns.

In some embodiments, the DNA binding domain is a catalytically-inactive Cas9 (dCas9) or a Cas9 nickase (nCas9). In some embodiments, the DNA binding domain is a catalytically-inactive Cpf1 (dCpf1).

Other aspects of the present disclosure provide information storage systems, including:

(i) a storage medium containing a plurality of nucleic acid molecules, each nucleic acid molecule containing one or more information storage regions containing a write address followed by a read address;

(ii) a modifying enzyme containing a DNA binding domain fused to a base editing enzyme that edits one or more target nucleotides in the write address of the plurality of nucleic acid molecules; and

(iii) a plurality of guide RNAs (gRNAs), each gRNA containing a specificity determining sequence (SDS) that is complementary to one type of information storage region in the plurality of nucleic acid molecules.

In some embodiments, the storage system is for use in storage of information in vitro.

In some embodiments, the DNA binding domain is a catalytically-inactive Cas9 (dCas9) or a Cas9 nickase (nCas9). In some embodiments, the DNA binding domain is a catalytically-inactive Cpf1 (dCpf1).

Other aspects of the present disclosure provide nucleic acid libraries containing a plurality of synthetic oligonucleotides, each oligonucleotide containing one or more information storage regions containing a write address followed by a read address.

In some embodiments, the write address contains one or more deoxycytidines or deoxyadenosines. In some embodiments, each oligonucleotide further contains a sequencing adaptor.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. For purposes of clarity, not every component may be labeled in every drawing.

FIG. 1 is a schematic showing a modifying enzyme (the cytidine-deaminase(CDA)-dCas9 fusion protein) using an address molecule (a guide RNA or gRNA) to target and modify (deaminate) specific deoxycytidines in a storage medium. Deamination of the deoxycytidine converts it to uridine, which is converted to thymidine after replication. The target sequence is specified by the gRNA sequence. The modifying enzyme can be retargeted to any desired sequence by changing the gRNA sequence.

FIG. 2 is a schematic showing a pool of oligonucleotides having unique memory address. The pool of oligonucleotides can be used as the storage medium described herein.

FIG. 3 shows the different types of storage mediums: a pool of oligonucleotides, a naturally occurring genome (self-replicating DNA such as bacterial genome), and a synthetic easily replicable DNA molecule (e.g., a plasmid).

FIGS. 4A-4B are schematics showing the process and results of high-throughput information recording and storage. (FIG. 4A) The storage strategy is analogous to punch cards, where a series of initially similar registers (lines/DNA oligonucleotides) in unmodified state (“0”) which can be flipped (punched/edited) to modified state (“1”) at specific addresses to store information. (FIG. 4B) High-throughput information storage.

FIG. 5 shows a repurposed “printer device” for printing the storage system components onto a support medium.

### DETAILED DESCRIPTION OF CERTAIN EMBODIMENTS

The present disclosure, in some aspects, provide systems and methods for in vitro information recording and storage using nucleic acids (e.g., DNA) as storage medium. A “storage medium” refers to a physical material that holds information. The storage medium described herein comprises a plurality of nucleic acid molecules (e.g., DNA molecules). The “information” to be stored are artificial or digital information, e.g., without limitation, books, movies, pictures, etc. Nucleic acids (e.g., DNA) are suitable as storage medium for long-term information storage due to its properties such as high encoding capacity and stability.

Methods of using DNA for recording digital information have been described in the art, all relying on DNA synthesis. However, with current DNA synthesis technologies, it is very costly to produce DNA in large scale to make information storage in DNA practical. Further, information storage by DNA synthesis requires the synthesis of a new storage medium every time new information need to be stored. The information storage systems described herein obviate the need for DNA synthesis and instead uses editing of clonal population of DNA molecules (such as plasmids that can be produced very cheaply) for information storage. Further, it is also much cheaper to record information using the methods described herein in the storage medium that have been produced in bulk than synthesizing a new storage medium for new information.

Components of the information storage system described herein include, in some embodiments, a storage medium comprising a plurality of nucleic acid molecules, a plurality of address molecules that target the nucleotides in the storage medium, and a modifying enzyme that uses the address molecules to target and modify the nucleotides in the storage medium. In some embodiments, the compositions and methods described herein can be used in a high throughput format, e.g., in conjunction with a “printer” (e.g., a printing device capable of printing on a surface, such as a (repurposed) inkjet printer) that is capable of spotting, with high resolution, the address molecule and modifying enzyme onto suitable support medium (e.g., paper) that has been preloaded with the storage medium. The recording of the information is carried out in vitro.

The storage medium of the present disclosure comprises a plurality of nucleic acid molecules, each nucleic acid molecule comprising one or more information storage regions, and each information storage region comprising a write address followed by a read address. A “nucleic acid” is at least two nucleotides covalently linked together, and in some instances, may contain phosphodiester bonds (e.g., a phosphodiester “backbone”). A nucleic acid may be DNA (e.g., genomic or episomal), RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine. Nucleic acids of the present disclosure may be produced using standard molecular biology methods (see, e.g., Green and Sambrook, Molecular Cloning, A Laboratory Manual, 2012, Cold Spring Harbor Press), isolated from an organism (e.g., bacteria), or synthesized de novo. For the purpose of the present disclosure, DNA (e.g., double stranded DNA) is a preferred storage medium at least due to its stability.

Each nucleic acid molecule in the storage medium described herein comprises one or more information storage regions. An “information storage region,” as described herein, refers to the regions in the nucleic acid molecule that is recognized, bound, and modified by the modifying enzyme. In some embodiments, each nucleic acid molecule in the storage medium comprises 1-10000 information storage regions. For example, each nucleic acid molecule in the storage medium may comprise 1-10000, 1-1000, 1-100, 1-10, 10-10000, 10-1000, 10-100, 100-10000, 100-1000, or 1000-10000 information storage regions. In some embodiments, each nucleic acid molecule in the storage medium comprises 1, 10, 20, 50, 100, 150, 200, 250, 300, 250, 400, 450, 500, 550, 600, 650, 700, 750, 800, 850, 900, 950, 1000, 2000, 3000, 4000, 5000, 6000, 7000, 8000, 9000, or 10000 information storage regions. In some embodiments, each nucleic acid molecule in the storage medium comprises more than 10000 information storage regions.

In some embodiments, the information storage region is 15-100 base pairs in length. For example, the information storage region may be 15-100, 20-100, 25-100, 30-100, 35-100, 40-100, 45-100, 50-100, 55-100, 60-100, 65-100, 70-100, 75-100, 80-100, 85-100, 90-100, 95-100, 15-95, 20-95, 25-95, 30-95, 35-95, 40-95, 45-95, 50-95, 55-95, 60-95, 65-95, 70-95, 75-95, 80-95, 85-95, 90-95, 15-90, 20-90, 25-90, 30-90, 35-90, 40-90, 45-90, 50-90, 55-90, 60-90, 65-90, 70-90, 75-90, 80-90, 85-90, 15-85, 20-85, 25-85, 30-85, 35-85, 40-85, 45-85, 50-85, 55-85, 60-85, 65-85, 70-85, 75-85, 80-85, 15-80, 20-80, 25-80, 30-80, 35-80, 40-80, 45-80, 50-80, 55-80, 60-80, 65-80, 70-80, 75-80, 15-75, 20-75, 25-75, 30-75, 35-75, 40-75, 45-75, 50-75, 55-75, 60-75, 65-75, 70-75, 15-70, 20-70, 25-70, 30-70, 35-70, 40-70, 45-70, 50-70, 55-70, 60-70, 65-70, 15-65, 20-65, 25-65, 30-65, 35-65, 40-65, 45-65, 50-65, 55-65, 60-65, 15-60, 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 base pairs in length. In some embodiments, the information storage region is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 based pairs in length. In some embodiments, the information storage region is more than 100 (e.g., 105, 110, 115, 120, or more) base pairs in length. In some embodiments, the information storage region is less than 15 (e.g., 10, 11, 12, 13, or 14) base pairs in length.

Each of the information storage regions comprises a write address followed by a read address. A “write address,” as used herein, refers to a region of the nucleic acid molecule that is modified by the modifying enzyme for information recording. The information is encoded in the modified nucleotide. As such, the write address contains nucleotides that is targeted and modified by the modifying enzyme, these nucleotides are termed herein as “target nucleotides.” If the nucleic acid molecule is a double stranded DNA molecule, the target nucleotide may be one or both the strands. The target nucleotide may be deoxycytidine (dC), deoxyadenosine (dA), deoxyguanosine (dG), or thymidine (also termed deoxythymidine, dT), depending on the strand it is one and depending on the modifying enzyme. In some embodiments, the write address comprises one or more (e.g., 1, 2, 3, 4, 5, 6, 7, 8, 9, 10, 11, 12, 13, 14, 15 or more) deoxycytidines. In some embodiments, the write address comprises one or more (e.g., 1, 2, 3, 4, 5, 6, 7, 8, 9, 10, 11, 12, 13, 14, 15 or more) deoxyadenosines.

It is to be noted that the write address is the region that is mostly likely to be modified by the modifying enzyme. It is possible for the modifying enzyme to modify nucleotides outside of the read address. Different modifying enzymes may also have different modifying windows, e.g., ranging from 1-20 base pairs. The modifying window of the modifying enzyme can also be tuned, e.g., by varying the length of the linker that is linking the different domains in the modifying enzyme.

In some embodiments, the write address is 5-40 base pairs in length. For example, the write address may be 5-40, 5-35, 5-30, 5-25, 5-20, 5-15, 5-10, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 base pairs in length. In some embodiments, the write address is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs in length.

In some embodiments, at least 20% of the nucleotides in the write address are target nucleotides. For example, at least 20%, at least 30%, at least 40%, at least 50%, at least 60%, at least 70%, at least 80%, at least 90%, or at least 95% of the nucleotides in the write address are target nucleotides. In some embodiments, 20%, 30%, 40%, 50%, 60%, 70%, 80%, 90%, or 100% of the nucleotides in the write address are target nucleotides.

The write address is followed by a read address. A “read address” is the region of the nucleic acid molecule that mediates the binding of the modifying enzyme. The write address is “followed by” the read address means that the read address is immediately downstream of (i.e., 3′ to) the write address or adjacent to (e.g., with less than 10, 9, 8, 7, 6, 5, 4, 3, or 2 base pairs in between) the write address on the 3′ side. In some embodiments, the read address is 10-60 base pairs in length. For example, the read address may be 10-60, 10-55, 10-50, 10-45, 10-40, 10-35, 10-30, 10-25, 10-20, 10-15, 15-60, 15-55, 15-50, 15-45, 15-40, 15-35, 15-30, 15-25, 15-20, 20-60, 20-55, 20-50, 20-45, 20-40, 20-35, 20-30, 20-25, 25-60, 25-55, 25-50, 25-45, 25-40, 25-35, 25-30, 30-60, 30-55, 30-50, 30-45, 30-40, 30-35, 35-60, 35-55, 35-50, 35-45, 35-40, 40-60, 40-55, 40-50, 40-45, 45-60, 45-55, 45-50, 50-60, 50-55, or 55-60 base pairs long. In some embodiments, the read address is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 base pairs long. In some embodiments, the read address is less than 10 base pairs in length. In some embodiments, the read address is more than 60 base pairs in length.

In some embodiments, the information storage region of the nucleic acid molecules in the storage medium comprises a Protospacer Adjacent Motif (PAM) immediately 3′ to an information storage region in the nucleic acid molecule. A “protospacer adjacent motif” (PAM) is typically a sequence of nucleotides located adjacent to (e.g., within 10, 9, 8, 7, 6, 5, 4, 3, 2, or 1) nucleotide(s) of a sequence that mediates the binding of a Cas9-based modifying enzyme (e.g., the read address in the information storage region). PAM is required for the activation of Cas9 nuclease domain, in the context of a wild-type Cas9. A PAM sequence is “immediately adjacent to” the information storage region if the PAM sequence is contiguous with the target sequence (that is, if there are no nucleotides located between the PAM sequence and the target sequence). In some embodiments, a PAM sequence is a wild-type PAM sequence. Examples of PAM sequences include, without limitation, NGG, NGR, NNGRR(T/N), NNNNGATT, NNAGAAW, NGGAG, and NAAAAC, AWG, CC. In some embodiments, a PAM sequence is obtained from Streptococcus pyogenes (e.g., NGG or NGR). In some embodiments, a PAM sequence is obtained from Staphylococcus aureus (e.g., NNGRR(T/N)). In some embodiments, a PAM sequence is obtained from Neisseria meningitidis (e.g., NNNNGATT). In some embodiments, a PAM sequence is obtained from Streptococcus thermophilus (e.g., NNAGAAW or NGGAG). In some embodiments, a PAM sequence is obtained from Treponema denticola NGGAG (e.g., NAAAAC). In some embodiments, a PAM sequence is obtained from Escherichia coli (e.g., AWG). In some embodiments, a PAM sequence is obtained from Pseudomonas auruginosa (e.g., CC). Other PAM sequences are contemplated. A PAM sequence is typically located downstream (i.e., 3′) from the target sequence, although in some embodiments a PAM sequence may be located upstream (i.e., 5′) from the target sequence.

In some embodiments, the information storage region of the nucleic acid molecules in the storage medium does not comprise a PAM. The PAM requirement for Cas9-based modifying enzyme may be bypassed by using a PAM-presenting oligonucleotide (PAMmer). A “PAM-presenting oligonucleotide (PAMmer)” refers to an oligonucleotide that contains a PAM sequence. It has been shown that providing a PAMmer in trans allows Cas9 to cleave RNA molecules that do not themselves contain a PAM sequence (e.g., as described in O'Connell et al., Nature, volume 516, pages 263-266, 2014; and Strutt et al., eLife, 7:e32724, 2018, incorporated herein by reference). The same strategy may be used herein for the modifying enzyme on nucleic acid molecules in the storage medium that do not contain a PAM sequence.

In some embodiments, the plurality of nucleic acid molecules in the storage medium are natural nucleic acids such as genomic DNA isolated from an organism. “Genomic DNA” refers to an organism's chromosomal DNA, in contrast to extra-chromosomal DNAs like plasmids. The genomic DNA of an organism (encoded by the genomic DNA) is the (biological) information of heredity which is passed from one generation of organism to the next. When genomic DNAs are used as the storage medium, unique information storage regions can be designated across the genomic DNA.

To be used as the storage medium of the present disclosure, the genomic DNA may be isolated from a range of organisms, including, without limitation, bacteria, viruses, and bacteriophages. Methods of isolating genomic DNAs are known to those skilled in the art.

Non-limiting examples of bacterial species whose genomic DNA can be used as the storage medium described herein include: Yersinia spp., Escherichia spp., Klebsiella spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Stremtomyces spp. In some embodiments, the bacterial cells are from Staphylococcus aureus, Bacillus subtilis, Clostridium butyricum, Brevibacterium lactofermentum, Streptococcus agalactiae, Lactococcus lactis, Leuconostoc lactis, Streptomyces, Actinobacillus actinobycetemcomitans, Bacteroides, cyanobacteria, Escherichia coli, Helobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus planta rum, Streptococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, Streptomyces ghanaenis, Halobacterium strain GRB, or Halobaferax sp. strain Aa2.2. In some embodiments, the storage medium is E. coli genomic DNA.

Non-limiting examples of viruses whose genomic DNA can be used as the storage medium described herein include: Herpesviruses, Caudoviruses, and Asfarviridae, Iridoviridae, Marseilleviridae, Mimiviridae, Phycodnaviridae, Poxviridae, Adenoviridae, Cortiviridae and Tectiviridae family viruses.

Non-limiting examples of bacteriophage whose genomic DNA can be used as the storage medium described herein include: 186 phage, λ phage, Φ6 phage, Φ29 phage, ΦX174, G4 phage, M13 phage, MS2 phage, N4 phage, P1 phage, P2 phage, P4 phage, R17 phage, T2 phage, T4 phage, T7 phage, and T12 phage.

In some embodiments, the genomic DNA is isolated from an eukaryotic cell (e.g., a yeast cell, an insect cell, or a mammalian cell such as a human cell).

In some embodiments, the plurality of nucleic acid molecules in the storage medium are plasmids. A “plasmid” is a small DNA molecule within a cell that is physically separated from a chromosomal DNA and can replicate independently. Plasmids are most commonly found as small circular, double-stranded DNA molecules in bacteria but are sometimes present in archaea and eukaryotic organisms. In nature, plasmids often carry genes that may benefit the survival of the organism, for example antibiotic resistance. While the chromosomes are big and contain all the essential genetic information for living under normal conditions, plasmids usually are very small and contain only additional genes that may be useful to the organism under certain situations or particular conditions. Artificial plasmids are widely used as vectors in molecular cloning, serving to drive the replication of recombinant DNA sequences within host organisms. Plasmids may be produced in large quantity with very low cost and shuttled in and out of cells and therefore are suitable for both in vitro and in vivo information storage. Plasmids can be engineered to contain all the requirement elements of the storage medium required (i.e., read address, write address, and PAM).

In some embodiments, the plurality of nucleic acid molecules in the storage medium are synthetic oligonucleotides. A “synthetic oligonucleotide” refers to a relatively short fragment of nucleic acids that is synthesized chemically. Synthetic oligonucleotides can be synthesized with any desired sequences. Methods of producing synthetic oligonucleotides are known to those skilled in the art.

In some embodiments, the synthetic oligonucleotides of the present disclosure are double stranded DNA molecules. In some embodiments, the synthetic oligonucleotides are 20-200 base pairs in length. For example, the synthetic oligonucleotides may be 20-200, 20-150, 20-100, 20-50, 50-200, 50-150, 50-100, 100-200, 100-150, or 150-200 base pairs long. In some embodiments, the synthetic oligonucleotides are 20, 30, 40, 50, 60, 70, 80, 90, 100, 110, 120, 130, 140, 150, 160, 170, 180, 190, or 200 base pairs long.

In some embodiments, a library of synthetic oligonucleotides may be synthesized, each carrying a different read address in the information storage region. For example, if the read address in the information storage region is n (n is an integer) base pairs in length, a total of 4ⁿ different synthetic oligonucleotides may be synthetized, each having a different read address. In some embodiments, n is at least 10 (e.g., 10, 11, 12, 13, 14, 15, 20, 25, 30, or more). In some embodiments, the write address of the synthetic oligonucleotides in the library comprises one or more (e.g., 1, 2, 3, 4, 5, 6, 7, 8, 9, 10, 11, 12, 13, 14, 15 or more) deoxycytidines. In some embodiments, the write address of the synthetic oligonucleotides in the library comprises one or more (e.g., 1, 2, 3, 4, 5, 6, 7, 8, 9, 10, 11, 12, 13, 14, 15 or more) deoxyadenosines.

One advantage of using synthetic oligonucleotides as storage medium is that sequencing adaptors can be appended to each of the synthetic oligonucleotides, facilitating reading out the recorded information via sequencing directly. Other types of storage medium (e.g., genomic DNA or plasmids) require more steps of preparation before sequencing can be carried out. A “sequence adaptor” refers to a short DNA sequence that can be appended to other DNA molecules to facilitate its sequencing using next generation sequencing techniques. Different adaptor sequences may be used for different nucleic acid molecules to be sequenced, facilitating their identification in the sequence results. The use of sequencing adaptors for next generation sequence, and adaptor sequences are known to those skilled in the art. Adaptors are also commercially available, e.g., from New England Biolabs or Illumina.

The information storage system described herein comprises a modifying enzyme that functions in recording information (i.e., making modifications in the storage medium). The modifying enzyme of the present disclosure comprises a DNA binding domain fused to a base editing enzyme. A “DNA binding domain,” as used herein, refers to a protein that binds to DNA in a sequence-specific manner. The DNA binding domain can direct the fused base editing enzyme to a target sequence to edit the target nucleotides. In some embodiments, the DNA binding domain is a RNA-guided nuclease. A “RNA-guided nuclease” refers to a nucleases with DNA binding specificity mediated by a guide nucleotide sequence (e.g., a gRNA). RNA-guided nucleases may be catalytically active (e.g., Cas9), catalytically inactive (e.g., dCas9), or catalytically partially active (e.g., Cas9 nickase or nCas9).

Non-limiting examples of RNA-guided endonucleases include Clustered regularly interspaced short palindromic repeats (CRISPR) associated protein 9 (Cas9) nucleases, e.g., Cas9 from Streptococcus pyogenes (e.g., as described in Jinek et al., Science 337:816-821(2012), incorporated herein by reference), and Cas9 from Prevotella and Francisella 1 (e.g., as described in Zetsche et al., Cell, 163, 759-771, 2015, incorporated herein by reference), and catalytically inactive or partially inactive variants thereof.

Cas9 nuclease sequences and structures are well known to those of skill in the art (see, e.g., Sanne et al., The CRISPR Journal, Vol. 1, No. 2, 2018; Ferretti et al., Proc. Natl. Acad. Sci. 98:4658-4663(2001); Deltcheva E. et al., Nature 471:602-607(2011); and Jinek et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., (2013) RNA Biology 10:5, 726-737; and Sanne et al., The CRISPR Journal, Vol. 1, No. 2, 2018, incorporated herein by reference.

In some embodiments, the RNA-guided endonuclease used herein is a Cas9 nuclease from Streptococcus pyogenes (Uniprot Reference Sequence: Q99ZW2). In some embodiments, Cas9 refers to a Cas9 from, without limitation: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1), Listeria innocua (NCBI Ref: NP 472073.1), Campylobacter jejuni (NCBI Ref: YP_002344900.1) or Neisseria meningitidis (NCBI Ref: YP_002342100.1).

In some embodiments, the RNA-guided nuclease is a Cas9 orthologue that is designated a different name, for example, the Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells.

In some embodiments, the RNA-guided nuclease in the modifying enzyme of the present disclosure is a catalytically-inactive Cas9 (dCas9) or Cas9 nickase (nCas9). The DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science 337:816-821(2012); Qi et al., Cell 28; 152(5):1173-83 (2013). In some embodiments, a partially inactive Cas9 (e.g., a Cas9 with one inactive DNA cleavage domain and one active DNA cleavage domain) is used as the RNA-guided DNA binding domain of the present disclosure. A partially inactive Cas9 cleaves one of the two DNA strands in the target sequence and is referred to herein as a “Cas9 nickase (nCas9).” In some embodiments, the nCas9 comprises an inactive RuvC domain. In some embodiments, the nCas9 comprises a D10A mutation that inactivates the RuvC domain.

In some embodiments, the RNA-guided nuclease in the modifying enzyme of the present disclosure is a catalytically inactive Cpf1 (dCpf1). The Cpf1 protein has a RuvC-like endonuclease domain that is similar to the RuvC domain of Cas9 but does not have a HNH endonuclease domain, and the N-terminal of Cpf1 does not have the alpha-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 (SEQ ID NO: 19) inactivates Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A in SEQ ID NO: 19. It is to be understood that any mutations, e.g., substitution mutations, deletions, or insertions that inactivates the RuvC domain of Cpf1 may be used in accordance with the present disclosure.

In some embodiments, the RNA guided nuclease is at least is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to any one of SEQ ID NOs: 15-24, and comprises the mutations that inactivates one or both of the nuclease domains.

A “base editing enzyme” is fused to the RNA guided nuclease to form the modifying enzyme used in the information storage system described herein. The base editing enzyme may be a cytidine deaminase or an adenosine deaminase. A “deaminase” refers to an enzyme that catalyzes the removal of an amine group from a molecule, or deamination, for example through hydrolysis.

In some embodiments, the deaminase is a cytidine deaminase. A “cytidine deaminase” refers to an enzyme that catalyzes the chemical reaction “cytosine+H₂O⇄NH₃” or “5-methyl-cytosine+H₂O⇄thymine+NH₃.”

One example of a suitable class of cytidine deaminases is the apolipoprotein B mRNA-editing complex (APOBEC) family of cytidine deaminases encompassing eleven proteins that serve to initiate mutagenesis in a controlled and beneficial manner. The apolipoprotein B editing complex 3 (APOBEC3) enzyme provides protection to human cells against a certain HIV-1 strain via the deamination of cytosines in reverse-transcribed viral ssDNA. These cytidine deaminases all require a Zn²⁺-coordinating motif (His-X-Glu-X₂₃₋₂₆-Pro-Cys-X₂₋₄-Cys) (SEQ ID NO: 51) and bound water molecule for catalytic activity. The glutamic acid residue acts to activate the water molecule to a zinc hydroxide for nucleophilic attack in the deamination reaction. Each family member preferentially deaminates at its own particular “hotspot,” for example, WRC (W is A or T, R is A or G) for hAID, or TTC for hAPOBEC3F. A recent crystal structure of the catalytic domain of APOBEC3G revealed a secondary structure comprising a five-stranded β-sheet core flanked by six α-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting. Another suitable cytidine deaminase is the activation-induced cytidine deaminase (AID), which is responsible for the maturation of antibodies by converting dCs in ssDNA to uracils in a transcription-dependent, strand-biased fashion.

Amino acid sequences of non-limiting, exemplary cytidine deaminases that may be used in accordance with the present disclosure are provided in Table 3. In some embodiments, the deaminase is a naturally-occurring deaminase from an organism, such as a human, chimpanzee, gorilla, monkey, cow, dog, rat, or mouse. In some embodiments, the deaminase is a variant of a naturally-occurring deaminase from an organism, and the variants do not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to any one of SEQ ID NOs: 25-47.

Cytidine deaminases catalyze the deamination of cytidine (C) to uridine (U), deoxycytidine (dC) to deoxyuridine (dU), or 5-methyl-cytidine to thymidine (T, 5-methyl-U), respectively. Subsequent DNA repair mechanisms ensure that a dU is replaced by T. DNA replication then converts the deoxyguanosine (dG) that is complementary to the dC to a dA, which complements the newly created thymidine (dT). Thus, effectively, the cytidine deaminase catalyzes the conversion of a dC:dG base pair to a dT:dA base pair in DNA.

Methods of introducing point mutations using a fusion protein comprising a RNA-guided nuclease (e.g., dCas9 or nCas9) fused to cytidine deaminase (e.g., APOBEC1) are known in the art (e.g., as described in Komor et al., Nature, 533, 420-424 (2016), incorporated herein by reference). In some embodiments, the editing efficiency of cytidine deaminases can be improved by fusing the uracil DNA glycosylase inhibitor (ugi) protein to the cytidine deaminase-dCas9/nCas9 fusion (e.g., also as described in Komor et al., Nature, 533, 420-424 (2016), incorporated herein by reference). When a cytidine deaminse-dCas9/nCas9 is used as the modifying enzyme, the write address of the nucleic acid molecules in the storage medium comprises one or more (e.g., 1, 2, 3, 4, 5, 6, 7, 8, 9, 10, 11, 12, 13, 14, 15, 16, 17, 18, 19, 20, or more) deoxycytidines.

In some embodiments, the base editing enzyme is an adenosine deaminase. An adenosine deaminase is an enzyme that catalyzes the deamination of adenosine to inosine. Adenosine deaminases catalyze the conversion of dA:dT base pairs to dG:dC base pairs. As described in Gaudelli et al. (Nature volume 551, pages 464-471, 2017, incorporated herein by reference), a transfer RNA adenosine deaminase was subjected to directed evolution and variants that can catalyze the deamination of deoxyadenosines in DNA were identified. These adenosine deaminase variants were also shown to be fused to dCas9 or nCas9 domains and used as modifying enzymes for nucleobase editing. These adenosine deaminase-dCas9/nCas9 fusion proteins can be used as the modifying enzymes of the present disclosure.

One skilled in the art is familiar with methods of making fusion proteins. Any linker sequences known in the art and described herein may be used for fusing the dCas9/nCas9 domain to the base editing enzyme. Varying the amino acid composition and the length of the linker may lead to different editing window of the modifying enzyme. In some embodiments, the dCas9/nCas9 is fused to the N-terminus of the base editing enzyme. In some embodiments, the dCas9/nCas9 domain is fused to the C-terminus of the base editing enzyme.

The modifying enzyme may be expressed using recombinant technology and purified for use in the systems and methods described herein. One skilled in the art is familiar with methods of expression and purifying recombinant proteins.

The information storage system described herein further comprises a plurality of address molecules. For modifying enzymes that contain RNA-guided nuclease domains, the address molecules are guide RNAs (gRNAs). The gRNAs for use as address molecules each comprises a specificity determining sequence (SDS) that is complementary to one type of information storage region in the plurality of nucleic acid molecules of the storage medium. The base modifying enzyme is targeted by the gRNAs to a target sequence (i.e., the information storage region for the purpose of the present disclosure), where it binds the target sequence and edits the target nucleotides. In some embodiments, each gRNA targets one type of information storage region in the nucleic acid molecules of the storage medium. The plurality of gRNAs may contain gRNAs that target all the different information storage regions (up to 4ⁿ types, wherein n is the length of the read address) in the plurality of nucleic acids in the storage medium.

A gRNA is a component of the CRISPR/Cas system. A “gRNA” (guide ribonucleic acid) herein refers to a fusion of a CRISPR-targeting RNA (crRNA) and a trans-activation crRNA (tracrRNA), providing both targeting specificity and scaffolding/binding ability for Cas9 nuclease. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences. The native gRNA comprises a 20 nucleotide (nt) Specificity Determining Sequence (SDS), which specifies the DNA sequence to be targeted, and is immediately followed by a 80 nt scaffold sequence, which associates the gRNA with Cas9. In some embodiments, an SDS of the present disclosure has a length of 15 to 100 nucleotides, or more. For example, an SDS may have a length of 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, or 15 to 20 nucleotides. In some embodiments, the SDS is 20 nucleotides long. For example, the SDS may be 15, 16, 17, 18, 19, 20, 21, 22, 23, 24, or 25 nucleotides long.

In some embodiments, at least a portion of the information storage region is complementary to the SDS of the gRNA. In some embodiments, an SDS is 100% complementary to the information storage region. In some embodiments, the SDS sequence is less than 100% complementary to the information storage region and is, thus, considered to be partially complementary. For example, the information storage region may be 99%, 98%, 97%, 96%, 95%, 94%, 93%, 92%, 91%, or 90% complementary the SDS of the gRNA. In some embodiments, the SDS of the gRNA may differ from the information storage region by 1, 2, 3, 4 or 5 nucleotides.

In addition to the SDS, the gRNA comprises a scaffold sequence (corresponding to the tracrRNA in the native CRISPR/Cas system) that is required for its association with Cas9 (referred to herein as the “gRNA handle”). In some embodiments, the gRNA comprises a structure 5′-[SDS]-[gRNA handle]-3′. In some embodiments, the scaffold sequence comprises the nucleotide sequence of 5′-guuuuagagcuagaaauagcaaguuaaaauaaaggcuaguc cguuaucaacuugaaaaaguggcaccgagucggugcuuuuu-3′ (SEQ ID NO: 50). Other non-limiting, suitable gRNA handle sequences that may be used in accordance with the present disclosure are listed in Table 1.

Further provided herein are methods of using the information storage system described herein for storing information. In some embodiments, the method comprises providing the storage medium described herein, and contacting, in vitro, the storage medium with the modifying enzyme and a plurality of gRNAs each comprising a SDS that is complementary to one type of information storage region in the plurality of nucleic acid molecules in the storage medium, wherein the contacting results in the editing of the one or more target nucleotides in the write address of the plurality of nucleic acid molecules.

In some embodiments, the modifying enzyme is a cytidine deaminase-dCas9/nCas9 fusion protein and the write address comprises one or more (e.g., 1, 2, 3, 4, 5, 6, 7, 8, 9, 10, 11, 12, 13, 14, 15, 16, 17, 18, 19, 20, or more) deoxycytidines. In some embodiments, the contacting results in a deoxycytidine to thymidine mutation on one strand. The deoxyguanosine that is complementary to the deoxycytosine on the other strand is changed to a deoxyadenosine in subsequent DNA replication. As such, the contacting results in a dC:dG base pair to dT:dA base pair conversion.

In some embodiments, the a modifying enzyme is an adenosine deaminase-dCas9/nCas9 fusion protein and the write address comprises one or more (e.g., 1, 2, 3, 4, 5, 6, 7, 8, 9, 10, 11, 12, 13, 14, 15, 16, 17, 18, 19, 20, or more) deoxyadenosines. In some embodiments, the contacting results in a deoxyadenosine to deoxyguanosine mutation on one strand. The thymidine that is complementary to the deoxyadenosine on the other strand is changed to a deoxycytosine in subsequent DNA replication. As such, the contacting results in a dA:dT base pair to dG:dC base pair conversion.

The information recorded in the storage medium can be read out by detecting the editing of the one or more target nucleotides in the write address. In some embodiments, the methods described herein further comprises detecting the editing of the one or more target nucleotides. In some embodiments, the detecting is via sequencing (e.g., next generation sequencing) of the nucleic acid molecules in the storage medium. In some embodiments, the information can be detected while it is being recorded in the nucleic acid molecules in the storage medium, e.g., using a technology similar to the Specific High-sensitivity Enzymatic Reporter unlocking (SHERLOCK) technology described in East-Seletsky et al., Nature volume 538, pages 270-273, 2016, incorporated herein by reference.

In some embodiments, higher-order and multiplex recording can be achieved, thus increasing the recording capacity. In some embodiments, encryption of the recorded information can be achieved. For example, both of these features can be achieved via executing ordered and combinations of DNA writing events in a controlled fashion. By carefully positioning the mutable residues in the gRNA SDS, the frequency and occurrence of DNA writing events can be controlled. The modifying enzyme can then be directed to desired information storage regions by providing complementary gRNAs. For example, two input AND logic operators can be built by layering two gRNAs that edit an information storage region. Once both edits are applied, the information storage region can be edited by a third RNA (e.g., to create a certain desired editing pattern), thus realizing the AND logic. Other logic operators can be made by providing different combinations of gRNAs and/or provide gRNAs in a specific order. In some embodiments, more efficient design could be achieved, by interconnecting DNA writing events and carefully designing sequence of DNA writing events.

In some embodiments, the method of recording information described herein can be carried out in a high-throughput manner and with spatial resolution. Being “high-throughput” means that at least 1000 (e.g., at least 1000, at least 2000, at least 3000, at least 4000, at least 5000, at least 6000, at least 7000, at least 8000, at least 9000, at least 10000, at least 100000, or more) recording events can occur at the same time. “Spatial resolution” means each of these recording events are occurring in its own separate space (i.e., not in the same reaction mix and is spatially separated).

For example, as illustrated in FIG. 5, a “printer-like device” or printing device can be used to spot the modifying enzyme and different combinations of gRNA and nucleic acid molecules in the storage medium onto an appropriate support medium (e.g., paper, film, etc.). In some embodiments, the storage medium (e.g., plasmids, genomic DNA, or synthetic oligonucleotides) and the modifying enzyme can be pre-spotted on a support medium and printing device (e.g., a repurposed inkjet printer) device can be used to deposit different combinations of gRNAs onto the support medium for information recording. In some embodiments, microfluidics devices can be used to add different combination of gRNAs to droplets containing the modifying enzyme and the storage medium, and the mixture can be spotted onto a support medium.

The “spotting” generates spatial resolution. Upon printing of modifying enzyme and gRNAs onto the support medium, information recording (i.e., editing of the DNA on the storage medium) occurs, generating different editing patterns at different spots of the support medium. “Editing pattern” refers to the number and position of the target nucleotides that are edited by the modifying enzyme in the write address of the nucleic acid molecules in the storage medium. Different combinations of gRNA and nucleic acid molecules in the different spots lead to different editing patterns. After information recording, the recorded storage medium can then be dried and stored. DNA can be stripped off the support medium and sequenced for information read out, when needed.

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teachings that are referenced herein.

### EXAMPLES

### Example 1. In Vitro Information Storage

The present disclosure, in some aspects, relates to in vitro DNA manipulation (e.g., base modifying) with nucleotide precision, rather than DNA synthesis for information storage in DNA. The DNA writing strategy is analogous to writing information on a piece of raw CD/hard drive, rather than making a new hard drive from scratch for every piece of information to be recorded. The cost of making lots of raw CD/hard drive is cheap, but making a new hard drive with a new set of information pre-written on it is expensive. To achieve this, a read/write head is needed to store information on unlimited number of cheaply obtainable raw CD/hard drives. The DNA writing strategy described herein, in some instances, can be used as a low-cost alternative for information storage in the absence of low-cost DNA synthesis technology.

The in vitro DNA writing system described herein comprises three components: storage medium, address molecules, and a modifying enzyme. The storage medium typically can be obtained in large quantities with low cost. Non-limiting examples of the storage medium include plasmids, a well-characterized genome (e.g., a bacterial genome or viral genome), or a synthetic oligonucleotide library. The address molecules are used to uniquely target the nucleotides in the storage medium. There's a one-time synthesis cost for these molecules, but once synthesized, the could be replicated with very low cost.

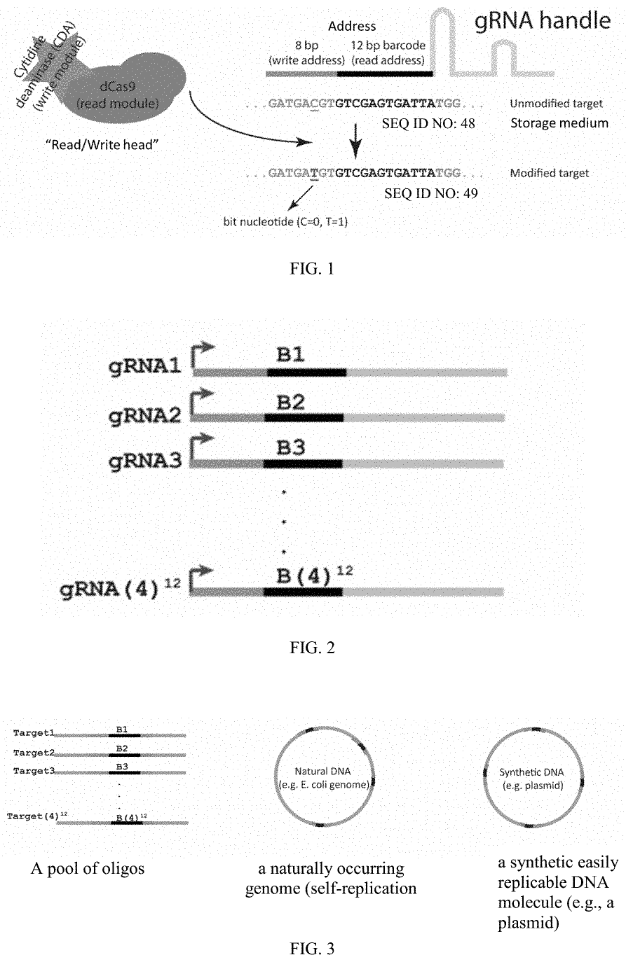

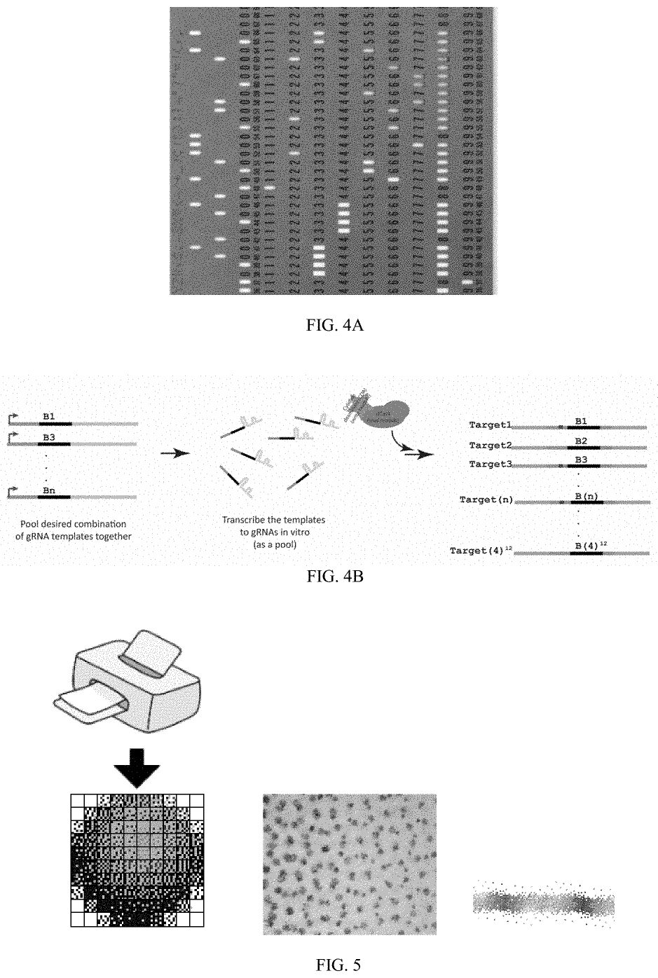

The modifying enzyme uses the address molecules to target and modify nucleotides in the storage medium. As demonstrated in FIG. 1, one example of the modifying enzyme is a cytidine-deaminase (CDA)-dCas9 fusion (Read/Write head) that use a gRNA (address) molecule to target and modify (i.e., deaminate) specific deoxycytidines (bit nucleotide) in a desired DNA molecule (storage medium) and mutate them to uridine, which are converted to thymidine after replication. The target sequence is specified by the gRNA sequence. The modifying enzyme can be easily retargeted to any desired sequence by changing the gRNA sequence.

The nucleic acids in the storage medium contains write and read addresses. The nucleotides that are targeted and edited by the modifying enzyme are in the write address, while the read address are used for the binding of the modifying enzyme, which is mediated by the gRNA. The read and write address may be of different lengths. When the read address is n (n is an integer) nucleotides long, a synthetic oligonucleotide library can contain up to 4ⁿ unique read addresses (FIG. 2). The up to 4ⁿ unique oligonucleotides can be synthesized and be used to produce gRNAs as templates in in vitro transcription reactions.

Different types of nucleic acid molecules can be used as the storage medium, e.g., genomic DNA, plasmids, and synthetic oligonucleotides (FIG. 3). Genomic DNA and plasmids could be produced in large quantity and with low cost. Plasmids can be designed to contained unique DNA addressed with all requirement (i.e., PAM domains and bit nucleotide(s) in correct positions). On the other hand, when using purified genomic DNA as a storage medium, unique memory registers can be designated across. Advantage of using a plasmid as memory register is that once information is stored, it can be easily shuttled in and out of cells for in vivo and in vitro information storage. Using a pooled library of oligonucleotides is more expensive but the advantage is that the storage medium with sequencing adaptors for fast readout by sequencing (other types of storage medium would require library prep before sequencing).

Cytidine deaminase (CDA)-dCas9 (the modifying enzyme) can be produced in large quantities by protein purification. A molecule of modifying enzyme can be used to modify many targets. CDA can be used to generate dC to dT as well as dG to dA mutations (depending which strand of DNA is targeted). Adenosine deaminase can be used instead of cytidine deaminase to modify dA and dT residues to dG and dC, respectively. Cas9 PAM requirement can be bypassed by using PAMMER (i.e., providing NGG in trans using oligonucleotides) to target sequences that lack a PAM domain. This strategy can be used to extend recording capacity when targeting a natural storage medium such as genomic DNA. Besides Cas9, other addressable DNA binding molecules (e.g., Cpf1 and Ago) can be fused to the writing module (cytidine/adenosine deaminases) which depending the application, could provide specific advantages.

Further, DNA information can be combined with various logic operators to achieve data encryption and higher-order and multiplex recording. For example, depending on the order and combinations that gRNAs are added, different outputs (i.e., editing patterns) can be achieved, thus increasing the recording capacity.

The recorded information can be read out offline (e.g., by sequencing), or online by a strategy similar to SHERLOCK (e.g., as described in East-Seletsky et al., Nature volume 538, pages 270-273, 2016, incorporated herein by reference).

The storage strategy is analogous to punch cards, where a series of initially similar registers (lines/DNA oligonucleotides) in unmodified state (“0”) which can be flipped (punched/edited) to modified state (“1”) at specific addresses to store information. Multiple memory registers can be designated and addressed in a single DNA molecule to increase the recording capacity to become more comparable with the recording capacity that can be achieved by DNA synthesis.

Ideally, with DNA writing technology, every single nucleotide in a storage medium can be addressed and edited, making the recording capacity of the approach comparable with DNA synthesis (in an ideal scenario, cytidine and adenosine deaminases as writer modules enable to achieve ˜50% of recording capacity that can be achieved by DNA synthesis).

In comparison to other information storage strategies based on DNA manipulation (such as oligo ligation strategies), the DNA writing strategy enables much higher recording capacity, as the system can be designed such that information can be recorded in every single base pair of the storage medium, whereas oligo ligation strategies require extensive of the DNA devoted to the invariable linkers and adaptors.

Unlike DNA ligation-based methods where bits of information (oligos) are recorded (ligated) sequentially in DNA, recoding information on a single storage medium molecule by DNA writing can be highly multiplexed and performed in a single pot by using a pool of gRNAs. Further, recording information by oligo ligation-based methods could generate extensive repeats which could eventually limit the ligation (i.e., recording) and sequencing (i.e., reading) capacity. Since information storage by DNA writing does not involve any repeat formation, higher information densities can be stored in DNA molecules and retrieval of information recorded by this method would be easier and more compatible with the current sequencing methods. Information can be directly encoded on a self-replicating genetic material (e.g. a plasmid) which can then be shuttled to cells for in vivo information storage.

A possible way to require spatial resolution required to make this a throughput technology is to use a printer-like device. Printing could be a cheap alternative to avoid cost of microfluidics/automation required for building a high-capacity information storage system. Instead of different color inks, such device can be used to spot (i.e., generate spatial separation) the gRNA and CDA-n/d-Cas9 (or lysate of cells expressing these components) along with storage medium on a paper (or any other suitable support medium). Upon printing, the editing occurs and the printed paper containing the recorded storage medium can then be dried and stored. DNA can be stripped off the paper and sequenced or replicated (e.g. by PCR) when necessary.

Current commercial printers can easily spot inks with resolution more than 1000 dpi. Even if the printing dpi is not very high or accurate for this specific purpose, Cas9 specificity should give enough discrimination in a given micro-environment to allow specific and targeted editing. Since multiple gRNAs can be used to edit multiple sites within one reaction, multiple gRNAs and targets can be combined within each dot printed by a printer thus increasing the throughput.

When using DNA writing to record information, any naturally available DNA that can be obtained cheaply and in large quantities (e.g., purified bacterial genome, plasmids) can be used as a storage medium, thus reducing the cost of information storage significantly. This addresses the major issue with oligo ligation-based methods since the cost of synthesis of oligonucleotides in huge quantities required for this method is still significant. Furthermore, after one-time synthesis of memory addresses (i.e., templates for gRNAs), unlimited quantities of the memory addresses can be produced enzymatically (by in vitro transcription) with a negligible cost.

The cost of microfluidics and automation to handle DNA manipulation reactions required for information storage is comparable between DNA synthesis and DNA manipulation-based methods (i.e., DNA writing and oligo ligation strategies).

It could be possible to lower the cost even more by using bacterial cells (and their lysates) to generate all the required components for DNA writing (i.e., plasmids as storage medium, CDA-dCas9 and gRNAs). To record different bits of information on a given plasmid, one would have to incubate the storage medium (e.g., a plasmid) with lysates of cells that express gRNAs and CDA-dCas9. This can be performed with a very low cost and in a high-throughput fashion.

### Example 2. Nucleotide Sequences and Amino Acid Sequences

Provided herein are exemplary guide RNA handle sequence (Table 1), exemplary RNA-guided nuclease sequences (Table 2), and exemplary cytidine deaminase sequences (Table 3).

TABLE 1
Exemplary Guide RNA Handle Sequences

| | | SEQ |
|---|---|---|
| | | ID |
| Organism | gRNA handle sequence | NO |
| S. pyogenes | GUUUAAGAGCUAUGCUGGAAAGCCACGGUGAA | 1 |
| | AAAGUUCAACUAUUGCCUGAUCGGAAUAAAUU | |
| | UGAACGAUACGACAGUCGGUGCUUUUUUU | |
| S. pyogenes | GUUUAAGAGCUAGAAAUAGCAAGUUUAAAUAA | 2 |
| | GGCUAGUCCGUUAUCAACUUGAAAAAGUGGCA | |
| | CCGAGUCGGUGCUUUUUU | |
| S. | GUUUUUGUACUCUCAAGAUUCAAUAAUCUUGC | 3 |
| thermophilus | AGAAGCUACAAAGAUAAGGCUUCAUGCCGAAA | |
| CRISPR1 | UCAACACCCUGUCAUUUUAUGGCAGGGUGUUU | |
| | U | |
| S. | GUUUUAGAGCUGUGUUGUUUGUUAAAACAACA | 4 |
| thermophilus | CAGCGAGUUAAAAUAAGGCUUAGUCCGUACUC | |
| CRISPR3 | AACUUGAAAAGGUGGCACCGAUUCGGUGUUUU | |
| | U | |
| C. jejuni | AAGAAAUUUAAAAAGGGACUAAAAUAAAGAGU | 5 |
| | UUGCGGGACUCUGCGGGGUUACAAUCCCCUAA | |
| | AACCGCUUUU | |
| F. novicida | AUCUAAAAUUAUAAAUGUACCAAAUAAUUAAU | 6 |
| | GCUCUGUAAUCAUUUAAAAGUAUUUUGAACGG | |
| | ACCUCUGUUUGACACGUCUGAAUAACUAAAA | |
| S. | UGUAAGGGACGCCUUACACAGUUACUUAAAUC | 7 |
| thermo- | UUGCAGAAGCUACAAAGAUAAGGCUUCAUGCC | |
| philus2 | GAAAUCAACACCCUGUCAUUUUAUGGCAGGGU | |
| | GUUUUCGUUAUUU | |
| M. mobile | UGUAUUUCGAAAUACAGAUGUACAGUUAAGAA | 8 |
| | UACAUAAGAAUGAUACAUCACUAAAAAAAGGC | |
| | UUUAUGCCGUAACUACUACUUAUUUUCAAAAU | |
| | AAGUAGUUUUUUUU | |
| L. innocua | AUUGUUAGUAUUCAAAAUAACAUAGCAAGUUA | 9 |
| | AAAUAAGGCUUUGUCCGUUAUCAACUUUUAAU | |
| | UAAGUAGCGCUGUUUCGGCGCUUUUUUU | |
| S. pyogenes | GUUGGAACCAUUCAAAACAGCAUAGCAAGUUA | 10 |
| | AAAUAAGGCUAGUCCGUUAUCAACUUGAAAAA | |
| | GUGGCACCGAGUCGGUGCUUUUUUU | |
| S. mutans | GUUGGAAUCAUUCGAAACAACACAGCAAGUUA | 11 |
| | AAAUAAGGCAGUGAUUUUUAAUCCAGUCCGUA | |
| | CACAACUUGAAAAAGUGCGCACCGAUUCGGUG | |
| | CUUUUUUAUUU | |
| S. | UUGUGGUUUGAAACCAUUCGAAACAACACAGC | 12 |
| thermophilus | GAGUUAAAAUAAGGCUUAGUCCGUACUCAACU | |
| | UGAAAAGGUGGCACCGAUUCGGUGUUUUUUUU | |
| N. | ACAUAUUGUCGCACUGCGAAAUGAGAACCGUU | 13 |
| meningitidis | GCUACAAUAAGGCCGUCUGAAAAGAUGUGCCG | |
| | CAACGCUCUGCCCCUUAAAGCUUCUGCUUUAA | |
| | GGGGCA | |
| P. multocida | GCAUAUUGUUGCACUGCGAAAUGAGAGACGUU | 14 |
| | GCUACAAUAAGGCUUCUGAAAAGAAUGACCGU | |
| | AACGCUCUGCCCCUUGUGAUUCUUAAUUGCAA | |
| | GGGGCAUCGUUUUU | |

TABLE 2
Exemplary Cas9 or Cas9 orthologue Sequences

| | | SEQ ID |
|---|---|---|
| Name | Sequence | NO: |
| S. pyogenes | MDKKYSIGLDIGTNSVGWAVITDEYKVPSKKFKVLGNTDRHSIKKNLIGALLF | 15 |
| Cas9 | DSGETAEATRLKRTARRRYTRRKNRICYLQEIFSNEMAKVDDSFFHRLEESFLV | |
| | EEDKKHERHPIFGNIVDEVAYHEKYPTIYHLRKKLVDSTDKADLRLIYLALAH | |
| | MIKFRGHFLIEGDLNPDNSDVDKLFIQLVQTYNQLFEENPINASGVDAKAILSA | |
| | RLSKSRRLENLIAQLPGEKKNGLFGNLIALSLGLTPNFKSNFDLAEDAKLQLSK | |
| | DTYDDDLDNLLAQIGDQYADLFLAAKNLSDAILLSDILRVNTEITKAPLSASMI | |
| | KRYDEHHQDLTLLKALVRQQLPEKYKEIFFDQSKNGYAGYIDGGASQEEFYKF | |
| | IKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRK | |
| | PAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDD | |
| | KVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVEN | |
| | TQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETN | |
| | GETGEIVWDKGRDFATVRKVLSMPQVNIVKKTEVQTGGFSKESILPKRNSDKL | |
| | IARKKDWDPKKYGGFDSPTVAYSVLVVAKVEKGKSKKLKSVKELLGITIMER | |
| | SSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDR | |
| | KRYTSTKEVLDATLIHQSITGLYETRIDLSQLGGD | |
| Francisella | MSIYQEFVNKYSLSKTLRFELIPQGKTLENIKARGLILDDEKRAKDYKKAKQII | 16 |
| novicida | DKYHQFFIEEILSSVCISEDLLQNYSDVYFKLKKSDDDNLQKDFKSAKDTIKKQ | |
| Cpf1 | ISEYIKDSEKFKNLFNQNLIDAKKGQESDLILWLKQSKDNGIELFKANSDITDID | |
| (Uniport | EALEIIKSFKGWTTYFKGFHENRKNVYSSNDIPTSIIYRIVDDNLPKFLENKAKY | |
| Reference | ESLKDKAPEAINYEQIKKDLAEELTFDIDYKTSEVNQRVFSLDEVFEIANFNNY | |
| Sequence: | LNQSGITKFNTIIGGKFVNGENTKRKGINEYINLYSQQINDKTLKKYKMSVLFK | |
| A0Q7Q2): | QILSDTESKSFVIDKLEDDSDVVTTMQSFYEQIAAFKTVEEKSIKETLSLLFDDL | |
| | KAQKLDLSKIYFKNDKSLTDLSQQVFDDYSVIGTAVLEYITQQIAPKNLDNPSK | |
| | KEQELIAKKTEKAKYLSLETIKLALEEFNKHRDIDKQCRFEEILANFAAIPMEFD | |
| | EIAQNKDNLAQISIKYQNQGKKDLLQASAEDDVKAIKDLLDQTNNLLHKLKIF | |
| | HISQSEDKANILDKDEHFYLVFEECYFELANIVPLYNKIRNYITQKPYSDEKFKL | |
| | NFENSTLANGWDKNKEPDNTAILFIKDDKYYLGVMNKKNNKIFDDKAIKENK | |
| | GEGYKKIVYKLLPGANKMLPKVFFSAKSIKFYNPSEDILRIRNHSTHTKNGSPQ | |
| | KGYEKFEFNIEDCRKFIDFYKQSISKHPEWKDFGFRFSDTQRYNSIDEFYREVE | |
| | NQGYKLTFENISESYIDSVVNQGKLYLFQIYNKDFSAYSKGRPNLHTLYWKAL | |
| | FDERNLQDVVYKLNGEAELFYRKQSIPKKITHPAKEAIANKNKDNPKKESVFE | |
| | YDLIKDKRFTEDKFFFHCPITINFKSSGANKFNDEINLLLKEKANDVHILSIDRG | |
| | ERHLAYYTLVDGKGNIIKQDTFNIIGNDRMKTNYHDKLAAIEKDRDSARKDW | |
| | KKINNIKEMKEGYLSQVVHEIAKLVIEYNAIVVFEDLNFGFKRGRFKVEKQVY | |
| | QKLEKMLIEKLNYLVFKDNEFDKTGGVLRAYQLTAPFETFKKMGKQTGIIYYV | |
| | PAGFTSKICPVTGFVNQLYPKYESVSKSQEFFSKFDKICYNLDKGYFEFSFDYK | |
| | NFGDKAAKGKWTIASFGSRLINFRNSDKNHNWDTREVYPTKELEKLLKDYSIE | |
| | YGHGECIKAAICGESDKKFFAKLTSVLNTILQMRNSKTGTELDYLISPVADVNG | |
| | NFFDSRQAPKNMPQDADANGAYHIGLKGLMLLGRIKNNQEGKKLNLVIKNEE | |
| | YFEFVQNRNN | |
| S. pyogenes | MDKKYSIGLAIGTNSVGWAVITDEYKVPSKKFKVLGNTDRHSIKKNLIGALLF | 17 |
| dCas9 | DSGETAEATRLKRTARRRYTRRKNRICYLQEIFSNEMAKVDDSFFHRLEESFLV | |
| (D10A and | EEDKKHERHPIFGNIVDEVAYHEKYPTIYHLRKKLVDSTDKADLRLIYLALAH | |
| H840A, | MIKFRGHFLIEGDLNPDNSDVDKLFIQLVQTYNQLFEENPINASGVDAKAILSA | |
| mutated | RLSKSRRLENLIAQLPGEKKNGLFGNLIALSLGLTPNFKSNFDLAEDAKLQLSK | |
| residues are | DTYDDDLDNLLAQIGDQYADLFLAAKNLSDAILLSDILRVNTEITKAPLSASMI | |
| underlined) | KRYDEHHQDLTLLKALVRQQLPEKYKEIFFDQSKNGYAGYIDGGASQEEFYKF | |
| | IKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRK | |
| | PAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDD | |
| | KVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVEN | |
| | TQLQNEKLYLYYLQNGRDMYVDQELDINRLSDYDVDAIVPQSFLKDDSIDNK | |
| | VLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETN | |
| | GETGEIVWDKGRDFATVRKVLSMPQVNIVKKTEVQTGGFSKESILPKRNSDKL | |
| | IARKKDWDPKKYGGFDSPTVAYSVLVVAKVEKGKSKKLKSVKELLGITIMER | |
| | SSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDR | |
| | KRYTSTKEVLDATLIHQSITGLYETRIDLSQLGGD | |
| S. pyogenes | MDKKYSIGLAIGTNSVGWAVITDEYKVPSKKFKVLGNTDRHSIKKNLIGALLF | 18 |
| Cas9 | DSGETAEATRLKRTARRRYTRRKNRICYLQEIFSNEMAKVDDSFFHRLEESFLV | |
| Nickase | EEDKKHERHPIFGNIVDEVAYHEKYPTIYHLRKKLVDSTDKADLRLIYLALAH | |
| (D10A, | MIKFRGHFLIEGDLNPDNSDVDKLFIQLVQTYNQLFEENPINASGVDAKAILSA | |
| mutation is | RLSKSRRLENLIAQLPGEKKNGLFGNLIALSLGLTPNFKSNFDLAEDAKLQLSK | |
| underlined | DTYDDDLDNLLAQIGDQYADLFLAAKNLSDAILLSDILRVNTEITKAPLSASMI | |
| | KRYDEHHQDLTLLKALVRQQLPEKYKEIFFDQSKNGYAGYIDGGASQEEFYKF | |
| | IKPILEKMDGTEELLVKLNREDLLRKQRTFDNGSIPHQIHLGELHAILRRQEDFY | |
| | PFLKDNREKIEKILTFRIPYYVGPLARGNSRFAWMTRKSEETITPWNFEEVVDK | |
| | GASAQSFIERMTNFDKNLPNEKVLPKHSLLYEYFTVYNELTKVKYVTEGMRK | |
| | PAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDD | |
| | KVMKQLKRRRYTGWGRLSRKLINGIRDKQSGKTILDFLKSDGFANRNFMQLI | |
| | HDDSLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHPVEN | |
| | TQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERG | |
| | GLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETN | |
| | GETGEIVWDKGRDFATVRKVLSMPQVNIVKKTEVQTGGFSKESILPKRNSDKL | |
| | IARKKDWDPKKYGGFDSPTVAYSVLVVAKVEKGKSKKLKSVKELLGITIMER | |
| | SSFEKNPIDFLEAKGYKEVKKDLIIKLPKYSLFELENGRKRMLASAGELQKGNE | |
| | LALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDR | |
| | KRYTSTKEVLDATLIHQSITGLYETRIDLSQLGGD | |
| Francisella | MSIYQEFVNKYSLSKTLRFELIPQGKTLENIKARGLILDDEKRAKDYKKAKQII | 19 |
| novicida | DKYHQFFIEEILSSVCISEDLLQNYSDVYFKLKKSDDDNLQKDFKSAKDTIKKQ | |
| dCpf1 | ISEYIKDSEKFKNLFNQNLIDAKKGQESDLILWLKQSKDNGIELFKANSDITDID | |
| (D917A, | EALEIIKSFKGWTTYFKGFHENRKNVYSSNDIPTSIIYRIVDDNLPKFLENKAKY | |
| mutation is | ESLKDKAPEAINYEQIKKDLAEELTFDIDYKTSEVNQRVFSLDEVFEIANFNNY | |
| underlined) | LNQSGITKFNTIIGGKFVNGENTKRKGINEYINLYSQQINDKTLKKYKMSVLFK | |
| | QILSDTESKSFVIDKLEDDSDVVTTMQSFYEQIAAFKTVEEKSIKETLSLLFDDL | |
| | KAQKLDLSKIYFKNDKSLTDLSQQVFDDYSVIGTAVLEYITQQIAPKNLDNPSK | |
| | KEQELIAKKTEKAKYLSLETIKLALEEFNKHRDIDKQCRFEEILANFAAIPMEFD | |
| | EIAQNKDNLAQISIKYQNQGKKDLLQASAEDDVKAIKDLLDQTNNLLHKLKIF | |
| | HISQSEDKANILDKDEHFYLVFEECYFELANIVPLYNKIRNYITQKPYSDEKFKL | |
| | NFENSTLANGWDKNKEPDNTAILFIKDDKYYLGVMNKKNNKIFDDKAIKENK | |
| | GEGYKKIVYKLLPGANKMLPKVFFSAKSIKFYNPSEDILRIRNHSTHTKNGSPQ | |
| | KGYEKFEFNIEDCRKFIDFYKQSISKHPEWKDFGFRFSDTQRYNSIDEFYREVE | |
| | NQGYKLTFENISESYIDSVVNQGKLYLFQIYNKDFSAYSKGRPNLHTLYWKAL | |
| | FDERNLQDVVYKLNGEAELFYRKQSIPKKITHPAKEAIANKNKDNPKKESVFE | |
| | YDLIKDKRFTEDKFFFHCPITINFKSSGANKFNDEINLLLKEKANDVHILSIARG | |
| | ERHLAYYTLVDGKGNIIKQDTFNIIGNDRMKTNYHDKLAAIEKDRDSARKDW | |
| | KKINNIKEMKEGYLSQVVHEIAKLVIEYNAIVVFEDLNFGFKRGRFKVEKQVY | |
| | QKLEKMLIEKLNYLVFKDNEFDKTGGVLRAYQLTAPFETFKKMGKQTGIIYYV | |
| | PAGFTSKICPVTGFVNQLYPKYESVSKSQEFFSKFDKICYNLDKGYFEFSFDYK | |
| | NFGDKAAKGKWTIASFGSRLINFRNSDKNHNWDTREVYPTKELEKLLKDYSIE | |
| | YGHGECIKAAICGESDKKFFAKLTSVLNTILQMRNSKTGTELDYLISPVADVNG | |
| | NFFDSRQAPKNMPQDADANGAYHIGLKGLMLLGRIKNNQEGKKLNLVIKNEE | |
| | YFEFVQNRNN | |
| Francisella | MSIYQEFVNKYSLSKTLRFELIPQGKTLENIKARGLILDDEKRAKDYKKAKQII | 20 |
| novicida | DKYHQFFIEEILSSVCISEDLLQNYSDVYFKLKKSDDDNLQKDFKSAKDTIKKQ | |
| dCpf1 | ISEYIKDSEKFKNLFNQNLIDAKKGQESDLILWLKQSKDNGIELFKANSDITDID | |
| (E1006A, | EALEIIKSFKGWTTYFKGFHENRKNVYSSNDIPTSIIYRIVDDNLPKFLENKAKY | |
| mutation is | ESLKDKAPEAINYEQIKKDLAEELTFDIDYKTSEVNQRVFSLDEVFEIANFNNY | |
| underlined) | LNQSGITKFNTIIGGKFVNGENTKRKGINEYINLYSQQINDKTLKKYKMSVLFK | |
| | QILSDTESKSFVIDKLEDDSDVVTTMQSFYEQIAAFKTVEEKSIKETLSLLFDDL | |
| | KAQKLDLSKIYFKNDKSLTDLSQQVFDDYSVIGTAVLEYITQQIAPKNLDNPSK | |
| | KEQELIAKKTEKAKYLSLETIKLALEEFNKHRDIDKQCRFEEILANFAAIPMIFD | |
| | EIAQNKDNLAQISIKYQNQGKKDLLQASAEDDVKAIKDLLDQTNNLLHKLKIF | |
| | HISQSEDKANILDKDEHFYLVFEECYFELANIVPLYNKIRNYITQKPYSDEKFKL | |
| | NFENSTLANGWDKNKEPDNTAILFIKDDKYYLGVMNKKNNKIFDDKAIKENK | |
| | GEGYKKIVYKLLPGANKMLPKVFFSAKSIKFYNPSEDILRIRNHSTHTKNGSPQ | |
| | KGYEKFEFNIEDCRKFIDFYKQSISKHPEWKDFGFRFSDTQRYNSIDEFYREVE | |
| | NQGYKLTFENISESYIDSVVNQGKLYLFQIYNKDFSAYSKGRPNLHTLYWKAL | |
| | FDERNLQDVVYKLNGEAELFYRKQSIPKKITHPAKEAIANKNKDNPKKESVFE | |
| | YDLIKDKRFTEDKFFFHCPITINFKSSGANKFNDEINLLLKEKANDVHILSIDRG | |
| | ERHLAYYTLVDGKGNIIKQDTFNIIGNDRMKTNYHDKLAAIEKDRDSARKDW | |
| | KKINNIKEMKEGYLSQVVHEIAKLVIEYNAIVVFADLNFGFKRGRFKVEKQVY | |
| | QKLEKMLIEKLNYLVFKDNEFDKTGGVLRAYQLTAPFETFKKMGKQTGIIYYV | |
| | PAGFTSKICPVTGFVNQLYPKYESVSKSQEFFSKFDKICYNLDKGYFEFSFDYK | |
| | NFGDKAAKGKWTIASFGSRLINFRNSDKNHNWDTREVYPTKELEKLLKDYSIE | |
| | YGHGECIKAAICGESDKKFFAKLTSVLNTILQMRNSKTGTELDYLISPVADVNG | |
| | NFFDSRQAPKNMPQDADANGAYHIGLKGLMLLGRIKNNQEGKKLNLVIKNEE | |
| | YFEFVQNRNN | |
| Francisella | MSIYQEFVNKYSLSKTLRFELIPQGKTLENIKARGLILDDEKRAKDYKKAKQII | 21 |
| novicida | DKYHQFFIEEILSSVCISEDLLQNYSDVYFKLKKSDDDNLQKDFKSAKDTIKKQ | |
| dCpf1 | ISEYIKDSEKFKNLFNQNLIDAKKGQESDLILWLKQSKDNGIELFKANSDITDID | |
| (D1255A, | EALEIIKSFKGWTTYFKGFHENRKNVYSSNDIPTSIIYRIVDDNLPKFLENKAKY | |
| mutation is | ESLKDKAPEAINYEQIKKDLAEELTFDIDYKTSEVNQRVFSLDEVFEIANFNNY | |
| underlined) | LNQSGITKFNTIIGGKFVNGENTKRKGINEYINLYSQQINDKTLKKYKMSVLFK | |
| | QILSDTESKSFVIDKLEDDSDVVTTMQSFYEQIAAFKTVEEKSIKETLSLLFDDL | |
| | KAQKLDLSKIYFKNDKSLTDLSQQVFDDYSVIGTAVLEYITQQIAPKNLDNPSK | |
| | KEQELIAKKTEKAKYLSLETIKLALEEFNKHRDIDKQCRFEEILANFAAIPMIFD | |
| | EIAQNKDNLAQISIKYQNQGKKDLLQASAEDDVKAIKDLLDQTNNLLHKLKIF | |
| | HISQSEDKANILDKDEHFYLVFEECYFELANIVPLYNKIRNYITQKPYSDEKFKL | |
| | NFENSTLANGWDKNKEPDNTAILFIKDDKYYLGVMNKKNNKIFDDKAIKENK | |
| | GEGYKKIVYKLLPGANKMLPKVFFSAKSIKFYNPSEDILRIRNHSTHTKNGSPQ | |
| | KGYEKFEFNIEDCRKFIDFYKQSISKHPEWKDFGFRFSDTQRYNSIDEFYREVE | |
| | NQGYKLTFENISESYIDSVVNQGKLYLFQIYNKDFSAYSKGRPNLHTLYWKAL | |
| | FDERNLQDVVYKLNGEAELFYRKQSIPKKITHPAKEAIANKNKDNPKKESVFE | |
| | YDLIKDKRFTEDKFFFHCPITINFKSSGANKFNDEINLLLKEKANDVHILSIDRG | |
| | ERHLAYYTLVDGKGNIIKQDTFNIIGNDRMKTNYHDKLAAIEKDRDSARKDW | |
| | KKINNIKEMKEGYLSQVVHEIAKLVIEYNAIVVFEDLNFGFKRGRFKVEKQVY | |
| | QKLEKMLIEKLNYLVFKDNEFDKTGGVLRAYQLTAPFETFKKMGKQTGIIYYV | |
| | PAGFTSKICPVTGFVNQLYPKYESVSKSQEFFSKFDKICYNLDKGYFEFSFDYK | |
| | NFGDKAAKGKWTIASFGSRLINFRNSDKNHNWDTREVYPTKELEKLLKDYSIE | |
| | YGHGECIKAAICGESDKKFFAKLTSVLNTILQMRNSKTGTELDYLISPVADVNG | |
| | NFFDSRQAPKNMPQDAAANGAYHIGLKGLMLLGRIKNNQEGKKLNLVIKNEE | |
| | YFEFVQNRNN | |
| Francisella | MSIYQEFVNKYSLSKTLRFELIPQGKTLENIKARGLILDDEKRAKDYKKAKQII | 22 |
| novicida | DKYHQFFIEEILSSVCISEDLLQNYSDVYFKLKKSDDDNLQKDFKSAKDTIKKQ | |
| dCpf1 | ISEYIKDSEKFKNLFNQNLIDAKKGQESDLILWLKQSKDNGIELFKANSDITDID | |
| (D917A/ | EALEIIKSFKGWTTYFKGFHENRKNVYSSNDIPTSIIYRIVDDNLPKFLENKAKY | |
| D1255A, | ESLKDKAPEAINYEQIKKDLAEELTFDIDYKTSEVNQRVFSLDEVFEIANFNNY | |
| mutations | LNQSGITKFNTIIGGKFVNGENTKRKGINEYINLYSQQINDKTLKKYKMSVLFK | |
| are | QILSDTESKSFVIDKLEDDSDVVTTMQSFYEQIAAFKTVEEKSIKETLSLLFDDL | |
| underlined) | KAQKLDLSKIYFKNDKSLTDLSQQVFDDYSVIGTAVLEYITQQIAPKNLDNPSK | |
| | KEQELIAKKTEKAKYLSLETIKLALEEFNKHRDIDKQCRFEEILANFAAIPMIFD | |
| | EIAQNKDNLAQISIKYQNQGKKDLLQASAEDDVKAIKDLLDQTNNLLHKLKIF | |
| | HISQSEDKANILDKDEHFYLVFEECYFELANIVPLYNKIRNYITQKPYSDEKFKL | |
| | NFENSTLANGWDKNKEPDNTAILFIKDDKYYLGVMNKKNNKIFDDKAIKENK | |
| | GEGYKKIVYKLLPGANKMLPKVFFSAKSIKFYNPSEDILRIRNHSTHTKNGSPQ | |
| | KGYEKFEFNIEDCRKFIDFYKQSISKHPEWKDFGFRFSDTQRYNSIDEFYREVE | |
| | NQGYKLTFENISESYIDSVVNQGKLYLFQIYNKDFSAYSKGRPNLHTLYWKAL | |
| | FDERNLQDVVYKLNGEAELFYRKQSIPKKITHPAKEAIANKNKDNPKKESVFE | |
| | YDLIKDKRFTEDKFFFHCPITINFKSSGANKFNDEINLLLKEKANDVHILSIARG | |
| | ERHLAYYTLVDGKGNIIKQDTFNIIGNDRMKTNYHDKLAAIEKDRDSARKDW | |
| | KKINNIKEMKEGYLSQVVHEIAKLVIEYNAIVVFEDLNFGFKRGRFKVEKQVY | |
| | QKLEKMLIEKLNYLVFKDNEFDKTGGVLRAYQLTAPFETFKKMGKQTGIIYYV | |
| | PAGFTSKICPVTGFVNQLYPKYESVSKSQEFFSKFDKICYNLDKGYFEFSFDYK | |
| | NFGDKAAKGKWTIASFGSRLINFRNSDKNHNWDTREVYPTKELEKLLKDYSIE | |
| | YGHGECIKAAICGESDKKFFAKLTSVLNTILQMRNSKTGTELDYLISPVADVNG | |
| | NFFDSRQAPKNMPQDAAANGAYHIGLKGLMLLGRIKNNQEGKKLNLVIKNEE | |
| | YFEFVQNRNN | |
| Francisella | MSIYQEFVNKYSLSKTLRFELIPQGKTLENIKARGLILDDEKRAKDYKKAKQII | 23 |
| novicida | DKYHQFFIEEILSSVCISEDLLQNYSDVYFKLKKSDDDNLQKDFKSAKDTIKKQ | |
| dCpf1 | ISEYIKDSEKFKNLFNQNLIDAKKGQESDLILWLKQSKDNGIELFKANSDITDID | |
| (E1006A/ | EALEIIKSFKGWTTYFKGFHENRKNVYSSNDIPTSIIYRIVDDNLPKFLENKAKY | |
| D1255A, | ESLKDKAPEAINYEQIKKDLAEELTFDIDYKTSEVNQRVFSLDEVFEIANFNNY | |
| mutations | LNQSGITKFNTIIGGKFVNGENTKRKGINEYINLYSQQINDKTLKKYKMSVLFK | |
| are | QILSDTESKSFVIDKLEDDSDVVTTMQSFYEQIAAFKTVEEKSIKETLSLLFDDL | |
| underlined) | KAQKLDLSKIYFKNDKSLTDLSQQVFDDYSVIGTAVLEYITQQIAPKNLDNPSK | |
| | KEQELIAKKTEKAKYLSLETIKLALEEFNKHRDIDKQCRFEEILANFAAIPMIFD | |
| | EIAQNKDNLAQISIKYQNQGKKDLLQASAEDDVKAIKDLLDQTNNLLHKLKIF | |
| | HISQSEDKANILDKDEHFYLVFEECYFELANIVPLYNKIRNYITQKPYSDEKFKL | |
| | NFENSTLANGWDKNKEPDNTAILFIKDDKYYLGVMNKKNNKIFDDKAIKENK | |
| | GEGYKKIVYKLLPGANKMLPKVFFSAKSIKFYNPSEDILRIRNHSTHTKNGSPQ | |
| | KGYEKFEFNIEDCRKFIDFYKQSISKHPEWKDFGFRFSDTQRYNSIDEFYREVE | |
| | NQGYKLTFENISESYIDSVVNQGKLYLFQIYNKDFSAYSKGRPNLHTLYWKAL | |
| | FDERNLQDVVYKLNGEAELFYRKQSIPKKITHPAKEAIANKNKDNPKKESVFE | |
| | YDLIKDKRFTEDKFFFHCPITINFKSSGANKFNDEINLLLKEKANDVHILSIDRG | |
| | ERHLAYYTLVDGKGNIIKQDTFNIIGNDRMKTNYHDKLAAIEKDRDSARKDW | |
| | KKINNIKEMKEGYLSQVVHEIAKLVIEYNAIVVFADLNFGFKRGRFKVEKQVY | |
| | QKLEKMLIEKLNYLVFKDNEFDKTGGVLRAYQLTAPFETFKKMGKQTGIIYYV | |
| | PAGFTSKICPVTGFVNQLYPKYESVSKSQEFFSKFDKICYNLDKGYFEFSFDYK | |
| | NFGDKAAKGKWTIASFGSRLINFRNSDKNHNWDTREVYPTKELEKLLKDYSIE | |
| | YGHGECIKAAICGESDKKFFAKLTSVLNTILQMRNSKTGTELDYLISPVADVNG | |
| | NFFDSRQAPKNMPQDAAANGAYHIGLKGLMLLGRIKNNQEGKKLNLVIKNEE | |
| | YFEFVQNRNN | |
| Francisella | MSIYQEFVNKYSLSKTLRFELIPQGKTLENIKARGLILDDEKRAKDYKKAKQII | 24 |
| novicida | DKYHQFFIEEILSSVCISEDLLQNYSDVYFKLKKSDDDNLQKDFKSAKDTIKKQ | |
| Cpf1 | ISEYIKDSEKFKNLFNQNLIDAKKGQESDLILWLKQSKDNGIELFKANSDITDID | |
| (D917A/ | EALEIIKSFKGWTTYFKGFHENRKNVYSSNDIPTSIIYRIVDDNLPKFLENKAKY | |
| E1006A/ | ESLKDKAPEAINYEQIKKDLAEELTFDIDYKTSEVNQRVFSLDEVFEIANFNNY | |
| D1255A, | LNQSGITKFNTIIGGKFVNGENTKRKGINEYINLYSQQINDKTLKKYKMSVLFK | |
| mutations | QILSDTESKSFVIDKLEDDSDVVTTMQSFYEQIAAFKTVEEKSIKETLSLLFDDL | |
| are | KAQKLDLSKIYFKNDKSLTDLSQQVFDDYSVIGTAVLEYITQQIAPKNLDNPSK | |
| underlined) | KEQELIAKKTEKAKYLSLETIKLALEEFNKHRDIDKQCRFEEILANFAAIPMIFD | |
| | EIAQNKDNLAQISIKYQNQGKKDLLQASAEDDVKAIKDLLDQTNNLLHKLKIF | |
| | HISQSEDKANILDKDEHFYLVFEECYFELANIVPLYNKIRNYITQKPYSDEKFKL | |
| | NFENSTLANGWDKNKEPDNTAILFIKDDKYYLGVMNKKNNKIFDDKAIKENK | |
| | GEGYKKIVYKLLPGANKMLPKVFFSAKSIKFYNPSEDILRIRNHSTHTKNGSPQ | |
| | KGYEKFEFNIEDCRKFIDFYKQSISKHPEWKDFGFRFSDTQRYNSIDEFYREVE | |
| | NQGYKLTFENISESYIDSVVNQGKLYLFQIYNKDFSAYSKGRPNLHTLYWKAL | |
| | FDERNLQDVVYKLNGEAELFYRKQSIPKKITHPAKEAIANKNKDNPKKESVFE | |
| | YDLIKDKRFTEDKFFFHCPITINFKSSGANKFNDEINLLLKEKANDVHILSIARG | |
| | ERHLAYYTLVDGKGNIIKQDTFNIIGNDRMKTNYHDKLAAIEKDRDSARKDW | |
| | KKINNIKEMKEGYLSQVVHEIAKLVIEYNAIVVFADLNFGFKRGRFKVEKQVY | |
| | QKLEKMLIEKLNYLVFKDNEFDKTGGVLRAYQLTAPFETFKKMGKQTGIIYYV | |
| | PAGFTSKICPVTGFVNQLYPKYESVSKSQEFFSKFDKICYNLDKGYFEFSFDYK | |
| | NFGDKAAKGKWTIASFGSRLINFRNSDKNHNWDTREVYPTKELEKLLKDYSIE | |
| | YGHGECIKAAICGESDKKFFAKLTSVLNTILQMRNSKTGTELDYLISPVADVNG | |
| | NFFDSRQAPKNMPQDAAANGAYHIGLKGLMLLGRIKNNQEGKKLNLVIKNEE | |
| | YFEFVQNRNN | |

TABLE 3
Exemplary Cytidine deaminases

| | | SEQ ID |
|---|---|---|
| Name | Sequence | NO |
| Human AID | MDSLLMNRRKFLYQFKNVRWAKGRRETYLCYVVKRRDSATSFSLDFGYL | 25 |
| | RNKNGCHVELLFLRYISDWDLDPGRCYRVTWFTSWSPCYDCARHVADFL | |
| | RGNPNLSLRIFTARLYFCEDRKAEPEGLRRLHRAGVQIAIMTFKDYFYCWN | |
| | TFVENHERTFKAWEGLHENSVRLSRQLRRILLPLYEVDDLRDAFRTLGL | |
| Mouse AID | MDSLLMKQKKFLYHFKNVRWAKGRHETYLCYVVKRRDSATSCSLDFGHL | 26 |
| | RNKSGCHVELLFLRYISDWDLDPGRCYRVTWFTSWSPCYDCARHVAEFLR | |
| | WNPNLSLRIFTARLYFCEDRKAEPEGLRRLHRAGVQIGIMTFKDYFYCWNT | |
| | FVENRERTFKAWEGLHENSVRLTRQLRRILLPLYEVDDLRDAFRMLGF | |
| Dog AID | MDSLLMKQRKFLYHFKNVRWAKGRHETYLCYVVKRRDSATSFSLDFGHL | 27 |
| | RNKSGCHVELLFLRYISDWDLDPGRCYRVTWFTSWSPCYDCARHVADFLR | |
| | GYPNLSLRIFAARLYFCEDRKAEPEGLRRLHRAGVQIAIMTFKDYFYCWNT | |
| | FVENREKTFKAWEGLHENSVRLSRQLRRILLPLYEVDDLRDAFRTLGL | |
| Bovine AID | MDSLLKKQRQFLYQFKNVRWAKGRHETYLCYVVKRRDSPTSFSLDFGHL | 28 |
| | RNKAGCHVELLFLRYISDWDLDPGRCYRVTWFTSWSPCYDCARHVADFL | |
| | RGYPNLSLRIFTARLYFCDKERKAEPEGLRRLHRAGVQIAIMTFKDYFYCW | |
| | NTFVENHERTFKAWEGLHENSVRLSRQLRRILLPLYEVDDLRDAFRTLGL | |
| Mouse | MGPFCLGCSHRKCYSPIRNLISQETFKFHFKNLGYAKGRKDTFLCYEVTRK | 29 |
| APOBEC-3 | DCDSPVSLHHGVFKNKDNIHAEICFLYWFHDKVLKVLSPREEFKITWYMS | |
| | WSPCFECAEQIVRFLATHHNLSLDIFSSRLYNVQDPETQQNLCRLVQEGAQ | |
| | VAAMDLYEFKKCWKKFVDNGGRRFRPWKRLLTNFRYQDSKLQEILRPCYI | |
| | PVPSSSSSTLSNICLTKGLPETRFCVEGRRMDPLSEEEFYSQFYNQRVKHLC | |
| | YYHRMKPYLCYQLEQFNGQAPLKGCLLSEKGKQHAEILFLDKIRSMELSQ | |
| | VTITCYLTWSPCPNCAWQLAAFKRDRPDLILHIYTSRLYFHWKRPFQKGLC | |
| | SLWQSGILVDVMDLPQFTDCWTNFVNPKRPFWPWKGLEIISRRTQRRLRRI | |
| | KESWGLQDLVNDFGNLQLGPPMS | |
| Rat APOBEC- | MGPFCLGCSHRKCYSPIRNLISQETFKFHFKNLRYAIDRKDTFLCYEVTRKD | 30 |
| 3 | CDSPVSLHHGVFKNKDNIHAEICFLYWFHDKVLKVLSPREEFKITWYMSW | |
| | SPCFECAEQVLRFLATHHNLSLDIFSSRLYNIRDPENQQNLCRLVQEGAQVA | |
| | AMDLYEFKKCWKKFVDNGGRRFRPWKKLLTNFRYQDSKLQEILRPCYIPV | |
| | PSSSSSTLSNICLTKGLPETRFCVERRRVHLLSEEEFYSQFYNQRVKHLCYY | |
| | HGVKPYLCYQLEQFNGQAPLKGCLLSEKGKQHAEILFLDKIRSMELSQVIIT | |
| | CYLTWSPCPNCAWQLAAFKRDRPDLILHIYTSRLYFHWKRPFQKGLCSLW | |
| | QSGILVDVMDLPQFTDCWTNFVNPKRPFWPWKGLEIISRRTQRRLHRIKES | |
| | WGLQDLVNDFGNLQLGPPMS | |
| Rhesus | MVEPMDPRTFVSNFNNRPILSGLNTVWLCCEVKTKDPSGPPLDAKIFQGKV | 31 |
| macaque | YSKAKYHPEMRFLRWFHKWRQLHHDQEYKVTWYVSWSPCTRCANSVAT | |
| APOBEC-3G | FLAKDPKVTLTIFVARLYYFWKPDYQQALRILCQKRGGPHATMKIMNYNE | |
| | FQDCWNKFVDGRGKPFKPRNNLPKHYTLLQATLGELLRHLMDPGTFTSNF | |
| | NNKPWVSGQHETYLCYKVERLHNDTWVPLNQHRGFLRNQAPNIHGFPKG | |
| | RHAELCFLDLIPFWKLDGQQYRVTCFTSWSPCFSCAQEMAKFISNNEHVSL | |
| | CIFAARIYDDQGRYQEGLRALHRDGAKIAMMNYSEFEYCWDTFVDRQGRP | |
| | FQPWDGLDEHSQALSGRLRAI | |
| Chimpanzee | MKPHFRNPVERMYQDTFSDNFYNRPILSHRNTVWLCYEVKTKGPSRPPLD | 32 |
| APOBEC-3G | AKIFRGQVYSKLKYHPEMRFFHWFSKWRKLHRDQEYEVTWYISWSPCTK | |
| | CTRDVATFLAEDPKVTLTIFVARLYYFWDPDYQEALRSLCQKRDGPRATM | |
| | KIMNYDEFQHCWSKFVYSQRELFEPWNNLPKYYILLHIMLGEILRHSMDPP | |
| | TFTSNFNNELWVRGRHETYLCYEVERLHNDTWVLLNQRRGFLCNQAPHK | |
| | HGFLEGRHAELCFLDVIPFWKLDLHQDYRVTCFTSWSPCFSCAQEMAKFIS | |
| | NNKHVSLCIFAARIYDDQGRCQEGLRTLAKAGAKISIMTYSEFKHCWDTFV | |
| | DHQGCPFQPWDGLEEHSQALSGRLRAILQNQGN | |
| Green monkey | MNPQIRNMVEQMEPDIFVYYFNNRPILSGRNTVWLCYEVKTKDPSGPPLD | 33 |
| APOBEC-3G | ANIFQGKLYPEAKDHPEMKFLHWFRKWRQLHRDQEYEVTWYVSWSPCTR | |
| | CANSVATFLAEDPKVTLTIFVARLYYFWKPDYQQALRILCQERGGPHATM | |
| | KIMNYNEFQHCWNEFVDGQGKPFKPRKNLPKHYTLLHATLGELLRHVMD | |
| | PGTFTSNFNNKPWVSGQRETYLCYKVERSHNDTWVLLNQHRGFLRNQAP | |
| | DRHGFPKGRHAELCFLDLIPFWKLDDQQYRVTCFTSWSPCFSCAQKMAKFI | |
| | SNNKHVSLCIFAARIYDDQGRCQEGLRTLHRDGAKIAVMNYSEFEYCWDT | |
| | FVDRQGRPFQPWDGLDEHSQALSGRLRAI | |
| Human | MKPHFRNTVERMYRDTFSYNFYNRPILSRRNTVWLCYEVKTKGPSRPPLD | 34 |
| APOBEC-3G | AKIFRGQVYSELKYHPEMRFFHWFSKWRKLHRDQEYEVTWYISWSPCTKC | |
| | TRDMATFLAEDPKVTLTIFVARLYYFWDPDYQEALRSLCQKRDGPRATMK | |
| | IMNYDEFQHCWSKFVYSQRELFEPWNNLPKYYILLHIMLGEILRHSMDPPT | |
| | FTFNFNNEPWVRGRHETYLCYEVERMTINDTWVLLNQRRGFLCNQAPHKH | |
| | GFLEGRHAELCFLDVIPFWKLDLDQDYRVTCFTSWSPCFSCAQEMAKFISK | |
| | NKHVSLCIFTARIYDDQGRCQEGLRTLAEAGAKISIMTYSEFKHCWDTFVD | |
| | HQGCPFQPWDGLDEHSQDLSGRLRAILQNQEN | |
| Human | MKPHFRNTVERMYRDTFSYNFYNRPILSRRNTVWLCYEVKTKGPSRPRLD | 35 |
| APOBEC-3F | AKIFRGQVYSQPEHHAEMCFLSWFCGNQLPAYKCFQITWFVSWTPCPDCV | |
| | AKLAEFLAEHPNVTLTISAARLYYYWERDYRRALCRLSQAGARVKIMDDE | |
| | EFAYCWENFVYSEGQPFMPWYKFDDNYAFLHRTLKEILRNPMEAMYPHIF | |
| | YFHFKNLRKAYGRNESWLCFTMEVVKHHSPVSWKRGVFRNQVDPETHCH | |
| | AERCFLSWFCDDILSPNTNYEVTWYTSWSPCPECAGEVAEFLARHSNVNLT | |
| | IFTARLYYFWDTDYQEGLRSLSQEGASVEIMGYKDFKYCWENFVYNDDEP | |
| | FKPWKGLKYNFLFLDSKLQEILE | |
| Human | MNPQIRNPMERMYRDTFYDNFENEPILYGRSYTWLCYEVKIKRGRSNLLW | 36 |
| APOBEC-3B | DTGVFRGQVYFKPQYHAEMCFLSWFCGNQLPAYKCFQITWFVSWTPCPDC | |
| | VAKLAEFLSEHPNVTLTISAARLYYYWERDYRRALCRLSQAGARVTIMDY | |
| | EEFAYCWENFVYNEGQQFMPWYKFDENYAFLHRTLKEILRYLMDPDTFTF | |
| | NFNNDPLVLRRRQTYLCYEVERLDNGTWVLMDQHMGFLCNEAKNLLCGF | |
| | YGRHAELRFLDLVPSLQLDPAQIYRVTWFISWSPCFSWGCAGEVRAFLQEN | |
| | THVRLRIFAARIYDYDPLYKEALQMLRDAGAQVSIMTYDEFEYCWDTFVY | |
| | RQGCPFQPWDGLEEHSQALSGRLRAILQNQGN | |
| Human | MNPQIRNPMKAMYPGTFYFQFKNLWEANDRNETWLCFTVEGIKRRSVVS | 37 |
| APOBEC-3C | WKTGVFRNQVDSETHCHAERCFLSWFCDDILSPNTKYQVTWYTSWSPCPD | |
| | CAGEVAEFLARHSNVNLTIFTARLYYFQYPCYQEGLRSLSQEGVAVEIMDY | |
| | EDFKYCWENFVYNDNEPFKPWKGLKTNFRLLKRRLRESLQ | |
| Human | MEASPASGPRHLMDPHIFTSNFNNGIGRHKTYLCYEVERLDNGTSVKMDQ | 38 |
| APOBEC-3A | HRGFLHNQAKNLLCGFYGRHAELRFLDLVPSLQLDPAQIYRVTWFISWSPC | |
| | FSWGCAGEVRAFLQENTHVRLRIFAARIYDYDPLYKEALQMLRDAGAQVS | |
| | IMTYDEFKHCWDTFVDHQGCPFQPWDGLDEHSQALSGRLRAILQNQGN | |
| Human | MALLTAETFRLQFNNKRRLRRPYYPRKALLCYQLTPQNGSTPTRGYFENK | 39 |
| APOBEC-3H | KKCHAEICFINEIKSMGLDETQCYQVTCYLTWSPCSSCAWELVDFIKAHDH | |
| | LNLGIFASRLYYHWCKPQQKGLRLLCGSQVPVEVMGFPKFADCWENFVD | |
| | HEKPLSFNPYKMLEELDKNSRAIKRRLERIKIPGVRAQGRYMDILCDAEV | |
| Human | MNPQIRNPMERMYRDTFYDNFENEPILYGRSYTWLCYEVKIKRGRSNLLW | 40 |
| APOBEC-3D | DTGVFRGPVLPKRQSNHRQEVYFRFENHAEMCFLSWFCGNRLPANRRFQI | |
| | TWFVSWNPCLPCVVKVTKFLAEHPNVTLTISAARLYYYRDRDWRWVLLR | |
| | LHKAGARVKIMDYEDFAYCWENFVCNEGQPFMPWYKFDDNYASLHRTL | |
| | KEILRNPMEAMYPHIFYFHFKNLLKACGRNESWLCFTMEVTKHHSAVFRK | |
| | RGVFRNQVDPETHCHAERCFLSWFCDDILSPNTNYEVTWYTSWSPCPECA | |
| | GEVAEFLARHSNVNLTIFTARLCYFWDTDYQEGLCSLSQEGASVKIMGYK | |
| | DFVSCWKNFVYSDDEPFKPWKGLQTNFRLLKRRLREILQ | |
| Human | MTSEKGPSTGDPTLRRRIEPWEFDVFYDPRELRKEACLLYEIKWGMSRKIW | 41 |
| APOBEC-1 | RSSGKNTTNHVEVNFIKKFTSERDFHPSMSCSITWFLSWSPCWECSQAIREF | |
| | LSRHPGVTLVIYVARLFWHMDQQNRQGLRDLVNSGVTIQIMRASEYYHC | |
| | WRNFVNYPPGDEAHWPQYPPLWMMLYALELHCIILSLPPCLKISRRWQNH | |
| | LTFFRLHLQNCHYQTIPPHILLATGLIHPSVAWR | |
| Mouse | MSSETGPVAVDPTLRRRIEPHEFEVFFDPRELRKETCLLYEINWGGRHSVW | 42 |
| APOBEC-1 | RHTSQNTSNHVEVNFLEKFTTERYFRPNTRCSITWFLSWSPCGECSRAITEF | |
| | LSRHPYVTLFIYIARLYHHTDQRNRQGLRDLISSGVTIQIMTEQEYCYCWRN | |
| | FVNYPPSNEAYWPRYPHLWVKLYVLELYCIILGLPPCLKILRRKQPQLTFFT | |
| | ITLQTCHYQRIPPHLLWATGLK | |
| Rat APOBEC- | MSSETGPVAVDPTLRRRIEPHEFEVFFDPRELRKETCLLYEINWGGRHSIWR | 43 |
| 1 | HTSQNTNKHVEVNFIEKFTTERYFCPNTRCSITWFLSWSPCGECSRAITEFLS | |
| | RYPHVTLFIYIARLYHHADPRNRQGLRDLISSGVTIQIMTEQESGYCWRNFV | |
| | NYSPSNEAHWPRYPHLWVRLYVLELYCIILGLPPCLNILRRKQPQLTFFTIA | |
| | LQSCHYQRLPPHILWATGLK | |
| Petromyzon | MTDAEYVRIHEKLDIYTFKKQFFNNKKSVSHRCYVLFELKRRGERRACFW | 44 |
| marinus CDA1 | GYAVNKPQSGTERGIHAEIFSIRKVEEYLRDNPGQFTINWYSSWSPCADCA | |
| (pmCDA1) | EKILEWYNQELRGNGHTLKIWACKLYYEKNARNQIGLWNLRDNGVGLNV | |
| | MVSEHYQCCRKIFIQSSHNQLNENRWLEKTLKRAEKRRSELSIMIQVKILHT | |
| | TKSPAV | |
| Human | MKPHFRNTVERMYRDTFSYNFYNRPILSRRNTVWLCYEVKTKGPSRPPLD | 45 |
| APOBEC3G | AKIFRGQVYSELKYHPEMRFFHWFSKWRKLHRDQEYEVTWYISWSPCTKC | |
| D316R_D317R | TRDMATFLAEDPKVTLTIFVARLYYFWDPDYQEALRSLCQKRDGPRATMK | |
| | IMNYDEFQHCWSKFVYSQRELFEPWNNLPKYYILLHIMLGEILRHSMDPPT | |
| | FTFNFNNEPWVRGRHETYLCYEVERMHNDTWVLLNQRRGFLCNQAPHKH | |
| | GFLEGRHAELCFLDVIPFWKLDLDQDYRVTCFTSWSPCFSCAQEMAKFISK | |
| | NKHVSLCIFTARIYRRQGRCQEGLRTLAEAGAKISIMTYSEFKHCWDTFVD | |
| | HQGCPFQPWDGLDEHSQDLSGRLRAILQNQEN | |
| Human | MDPPTFTFNFNNEPWVRGRHETYLCYEVERMHNDTWVLLNQRRGFLCNQ | 46 |
| APOBEC3G | APHKHGFLEGRHAELCFLDVIPFWKLDLDQDYRVTCFTSWSPCFSCAQEM | |
| chain A | AKFISKNKHVSLCIFTARIYDDQGRCQEGLRTLAEAGAKISIMTYSEFKHCW | |
| | DTFVDHQGCPFQPWDGLDEHSQDLSGRLRAILQ | |
| Human | MDPPTFTFNFNNEPWVRGRHETYLCYEVERMHNDTWVLLNQRRGFLCNQ | 47 |
| APOBEC3G | APHKHGFLEGRHAELCFLDVIPFWKLDLDQDYRVTCFTSWSPCFSCAQEM | |
| chain A | AKFISKNKHVSLCIFTARIYRRQGRCQEGLRTLAEAGAKISIMTYSEFKHCW | |
| D120R_D121R | DTFVDHQGCPFQPWDGLDEHSQDLSGRLRAILQ | |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of storing information, comprising:
(i) providing a storage medium comprising a plurality of nucleic acid molecules, each nucleic acid molecule comprising one or more information storage regions, each information storage region comprising a write address followed by a read address; and
(ii) contacting, in vitro, the storage medium with:
(a) a modifying enzyme comprising a DNA binding domain fused to a base editing enzyme that edits one or more target nucleotides in the write address of the plurality of nucleic acid molecules, and
(b) a plurality of guide RNAs (gRNAs), each gRNA comprising a specificity determining sequence (SDS) that is complementary to one type of information storage region in the plurality of nucleic acid molecules;
wherein the contacting results in the editing of the one or more target nucleotides in the write address of the plurality of nucleic acid molecules.

2. The method of claim 1, wherein the DNA binding domain is a catalytically-inactive Cas9 (dCas9) or a Cas9 nickase (nCas9).

3. The method of claim 1, wherein the DNA binding domain is a catalytically-inactive Cpf1 (dCpf1).

4. The method of claim 1, wherein the plurality of nucleic acid molecules are isolated genomic DNA molecules, plasmids, or synthetic oligonucleotides.

5.-8. (canceled)

9. The method of claim 1, wherein each of the plurality of nucleic acid molecules further comprises a protospacer adjacent motif (PAM) following each information storage region.

10. The method of claim 1, wherein the plurality of nucleic acid molecules do not each comprise a PAM following each information storage region, and the method further comprises contacting the storage medium with a PAM-presenting oligonucleotide (PAMmer).

11. The method of claim 1, wherein the base editing enzyme is a cytidine deaminase and the write address comprises one or more deoxycytidines.

12. (canceled)

13. The method of claim 1, wherein the base editing enzyme is an adenosine deaminase and the write address comprises one or more deoxyadenosines.

14. (canceled)

15. The method of claim 1, wherein the method is carried out in a high-throughput manner.

16. The method of claim 1, further comprising:
(iii) detecting the editing of the one or more target nucleotides.

17. (canceled)

18. A method of storing information, comprising:
(i) providing a support medium comprising a plurality of spots, each spot containing a storage medium comprising a plurality of nucleic acid molecules, each nucleic acid molecule comprising one or more information storage regions, each information storage region comprising a write address followed by a read address, wherein different spots have different nucleic acid molecules; and
(ii) depositing using a printing device onto the plurality of spots on the support medium:
(a) a modifying enzyme comprising a DNA binding domain fused to a base editing enzyme that edits one or more target nucleotides in the write address of the plurality of nucleic acid molecules, and
(b) a plurality of guide RNAs (gRNAs), each gRNA comprising a specificity determining sequence (SDS) that is complementary to one type of information storage region in the plurality of nucleic acid molecules, wherein the gRNA deposited onto each spot is different;
wherein the contacting results in the editing of the one or more target nucleotides in the write address of the plurality of nucleic acid molecules, and wherein nucleic acid molecules in different spots have different editing patterns.

19. The method of claim 18, wherein the DNA binding domain is a catalytically-inactive Cas9 (dCas9) or a Cas9 nickase (nCas9).

20. The method of claim 18, wherein the DNA binding domain is a catalytically-inactive Cpf1 (dCpf1).

21. An information storage system, comprising:
(i) a storage medium comprising a plurality of nucleic acid molecules, each nucleic acid molecule comprising one or more information storage regions comprising a write address followed by a read address;
(ii) a modifying enzyme comprising a DNA binding domain fused to a base editing enzyme that edits one or more target nucleotides in the write address of the plurality of nucleic acid molecules; and
(iii) a plurality of guide RNAs (gRNAs), each gRNA comprising a specificity determining sequence (SDS) that is complementary to one type of information storage region in the plurality of nucleic acid molecules.

22. The information storage system of claim 21, for use in storage of information in vitro.

23. The information storage system of claim 21, wherein the DNA binding domain is a catalytically-inactive Cas9 (dCas9) or a Cas9 nickase (nCas9).

24. The information storage system of claim 21, wherein the DNA binding domain is a catalytically-inactive Cpf1 (dCpf1).

25. A nucleic acid library comprising a plurality of synthetic oligonucleotides, each oligonucleotide comprising one or more information storage regions comprising a write address followed by a read address.

26. The nucleic acid library of claim 25, wherein the write address comprises one or more deoxycytidines or deoxyadenosines.

27. The nucleic acid library of claim 25, wherein each oligonucleotide further comprises a sequencing adaptor.


SEQUENCE LISTING (51 sequences)

SEQ ID NO: 1 - RNA, 93 nt, Streptococcus pyogenes
guuuaagagcuaugcuggaaagccacggugaaaaaguucaacuauugccugaucggaauaaauuugaacgauacgacagucggugcuuuuuuu

SEQ ID NO: 2 - RNA, 82 nt, Streptococcus pyogenes
guuuaagagcuagaaauagcaaguuuaaauaaggcuaguccguuaucaacuugaaaaaguggcaccgagucggugcuuuuuu

SEQ ID NO: 3 - RNA, 97 nt, Artificial Sequence
  Synthetic Polynucleotide
guuuuuguacucucaagauucaauaaucuugcagaagcuacaaagauaaggcuucaugccgaaaucaacacccugucauuuuauggcaggguguuuu

SEQ ID NO: 4 - RNA, 97 nt, Artificial Sequence
  Synthetic Polynucleotide
guuuuagagcuguguuguuuguuaaaacaacacagcgaguuaaaauaaggcuuaguccguacucaacuugaaaagguggcaccgauucgguguuuuu

SEQ ID NO: 5 - RNA, 74 nt, Campylobacter jejuni
aagaaauuuaaaaagggacuaaaauaaagaguuugcgggacucugcgggguuacaauccccuaaaaccgcuuuu

SEQ ID NO: 6 - RNA, 95 nt, Francisella novicida
aucuaaaauuauaaauguaccaaauaauuaaugcucuguaaucauuuaaaaguauuuugaacggaccucuguuugacacgucugaauaacuaaaa

SEQ ID NO: 7 - RNA, 109 nt, Streptococcus thermophilus
uguaagggacgccuuacacaguuacuuaaaucuugcagaagcuacaaagauaaggcuucaugccgaaaucaacacccugucauuuuauggcaggguguuuucguuauuu

SEQ ID NO: 8 - RNA, 110 nt, Mycoplasma mobile
uguauuucgaaauacagauguacaguuaagaauacauaagaaugauacaucacuaaaaaaaggcuuuaugccguaacuacuacuuauuuucaaaauaaguaguuuuuuuu

SEQ ID NO: 9 - RNA, 92 nt, Listeria innocua
auuguuaguauucaaaauaacauagcaaguuaaaauaaggcuuuguccguuaucaacuuuuaauuaaguagcgcuguuucggcgcuuuuuuu

SEQ ID NO: 10 - RNA, 89 nt, Streptococcus pyogenes
guuggaaccauucaaaacagcauagcaaguuaaaauaaggcuaguccguuaucaacuugaaaaaguggcaccgagucggugcuuuuuuu

SEQ ID NO: 11 - RNA, 107 nt, Streptococcus mutans
guuggaaucauucgaaacaacacagcaaguuaaaauaaggcagugauuuuuaauccaguccguacacaacuugaaaaagugcgcaccgauucggugcuuuuuuauuu

SEQ ID NO: 12 - RNA, 96 nt, Streptococcus thermophilus
uugugguuugaaaccauucgaaacaacacagcgaguuaaaauaaggcuuaguccguacucaacuugaaaagguggcaccgauucgguguuuuuuuu

SEQ ID NO: 13 - RNA, 102 nt, Neisseria meningitidis
acauauugucgcacugcgaaaugagaaccguugcuacaauaaggccgucugaaaagaugugccgcaacgcucugccccuuaaagcuucugcuuuaaggggca

SEQ ID NO: 14 - RNA, 110 nt, Pasteurella multocida
gcauauuguugcacugcgaaaugagagacguugcuacaauaaggcuucugaaaagaaugaccguaacgcucugccccuugugauucuuaauugcaaggggcaucguuuuu

SEQ ID NO: 15 - protein, 1368 aa, Artificial Sequence [sequence omitted]

SEQ ID NO: 16 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 17 - protein, 1368 aa, Artificial Sequence
  Synthetic Polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 18 - protein, 1368 aa, Artificial Sequence
  Synthetic Polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 19 - protein, 1300 aa, Artificial Sequence
  Synthetic Polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 20 - protein, 1300 aa, Artificial Sequence
  Synthetic Polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 21 - protein, 1300 aa, Artificial Sequence
  Synthetic Polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 22 - protein, 1300 aa, Artificial Sequence
  Synthetic Polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 23 - protein, 1300 aa, Artificial Sequence
  Synthetic Polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 24 - protein, 1300 aa, Artificial Sequence
  Synthetic Polypeptide
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 25 - protein, 198 aa, Homo sapiens [sequence omitted]

SEQ ID NO: 26 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 27 - protein, 198 aa, Canis lupus familiaris
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 28 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 29 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 30 - protein, 429 aa, Rattus norvegicus
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 31 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 32 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 33 - protein, 377 aa, Chlorocebus aethiops
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 34 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 35 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 36 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 37 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 38 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 39 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 40 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 41 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 42 - protein, 229 aa, Homo sapiens
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 43 - protein, 229 aa, Rattus norvegicus [sequence omitted]

SEQ ID NO: 44 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 45 - protein, 384 aa, Artificial Sequence
  Synthetic Polypeptide
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQNQEN

SEQ ID NO: 46 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQ

SEQ ID NO: 47 - protein, 184 aa, Artificial Sequence
  Synthetic Polypeptide
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQ

SEQ ID NO: 48 - DNA, 23 bp, Artificial Sequence
  Synthetic Polynucleotide
gatgacgtgtcgagtgattatgg

SEQ ID NO: 49 - DNA, 23 bp, Artificial Sequence
  Synthetic Polynucleotide
gatgatgtgtcgagtgattatgg

SEQ ID NO: 50 - RNA, 82 nt, Artificial Sequence
  Synthetic Polynucleotide
guuuuagagcuagaaauagcaaguuaaaauaaaggcuaguccguuaucaacuugaaaaaguggcaccgagucggugcuuuuu

SEQ ID NO: 51 - protein, 36 aa, Artificial Sequence
  Synthetic Polypeptide
  misc_feature (2)..(2): Xaa can be any naturally occurring amino acid
  misc_feature (4)..(29): Xaa can be any naturally occurring amino acid
  MISC_FEATURE (27)..(29): may be absent
  misc_feature (32)..(35): Xaa can be any naturally occurring amino acid
  MISC_FEATURE (34)..(35): may be absent
HXEXXXXXXXXXXXXXXXXXXXXXXXXXXPCXXXXC